SEQUENCE LISTING (5 sequences)

SEQ ID NO: 1 - protein, 9 aa, Homo sapiens
GEFLRTSCG

SEQ ID NO: 2 - protein, 9 aa, Homo sapiens
GNFLKTSCG

SEQ ID NO: 3 - protein, 9 aa, Homo sapiens
SDSLTTSTP

SEQ ID NO: 4 - protein, 9 aa, Homo sapiens
IFGLVTNLE

SEQ ID NO: 5 - protein, 9 aa, Homo sapiens
GNLLLTTGG

(19) United States
(12) Patent Application Publication
(10) Pub. No.: US 2012/0122991 A1
(43) Pub. Date: May 17, 2012

(54) METHOD FOR TREATING CANCER BY INCREASING AMP-ACTIVATED KINASE ACTIVITY

(75) Inventors: Lewis C. Cantley, Cambridge, MA (US); Reuben J. Shaw, San Diego, CA (US); Nabeel Bardeesy, Boston, MA (US); Ronald A. Depinho, Brookline, MA (US)

(73) Assignees: Dana-Farber Cancer Institute, Inc., Boston, MA (US); Beth Israel Deaconess Medical Center, Inc., Boston, MA (US)

(21) Appl. No.: 13/354,953

(22) Filed: Jan. 20, 2012

Related U.S. Application Data
(63) Continuation of application No. 10/571,242, filed on Apr. 30, 2007.
(60) Provisional application No. 60/501,503, filed on Sep. 6, 2003.
(60) Provisional application No. 60/506,705, filed on Sep. 26, 2003.

(51) Int. Cl. A61K 31/155; A61P 35/00

(52) U.S. Cl. 514/635

(57) ABSTRACT

The invention relates to modulation of LKB1 or AMP kinase protein activity for treating disorders including diabetes and cancer. The invention also relates to screening for agents that modulate the activity of LKB1 or AMP kinase protein, which are useful in the treatment of diabetes and cancer, as well as preparing compounds for treatment of diabetes and cancer.

### RELATED APPLICATIONS

This application is a Continuation of U.S. application Ser. No. 10/571,242, filed on Sep. 9, 2004, which is a national stage filing under 35 U.S.C. §371 of international application PCT/US2004/029437, filed Sep. 9, 2004, which was published under PCT Article 21(2) in English, which claims of the benefit under 35 U.S.C. §119(e) of U.S. Provisional Application No. 60/501,513, filed Sep. 9, 2003 and U.S. Provisional Application No. 60/506,705, filed Sep. 26, 2003, the entire contents of each of which are incorporated herein by reference.

### GOVERNMENT SUPPORT

This invention was made in part with government support under grant number(s) GM56203 (5R01) from the National Institutes of Health (N1H). The government may have certain rights in this invention.

### FIELD OF THE INVENTION

The invention relates to modulation of LKB1 or AMP kinase protein activity for treating disorders including diabetes and cancer. The invention also relates to screening for agents that modulate the activity of LKB1 or AMP kinase protein, which are useful in the treatment of diabetes and cancer, as well as preparing compounds for treatment of diabetes and cancer.

### BACKGROUND OF THE INVENTION

An estimated 15.7 million Americans have diabetes, and individuals with adult-onset, type II, diabetes represent 90 to 95 percent of all diabetics. Almost one-third of all diabetics in the U.S. are unaware that they have the disorder, and undetected and uncontrolled diabetes can have serious side effects, such as blindness, heart disease, nerve disease, and kidney disease.

Impaired energy metabolism is a primary defect in type 2 diabetes (Rutter, G. A., et al., 2003 Biochem J. 375 (Pt 1):1-16). AMP-activated protein kinase (AMPK) is a highly conserved sensor of cellular energy status found in all eukaryotic cells (Hardie, D. G., et al., 2003 FEBS Letters 546, 1113-120). Recent studies have indicated that AMPK is a critical regulator of leptin-induced fatty acid metabolism and glucose uptake in skeletal muscle (Minokoshi, Y., et al., 2002. Nature 415, 339-43; Mu, J., et al., 2001 Mol. Cell. 7, 1085-94). AMPK is activated by stimuli that increase ATP consumption or inhibit ATP production in mammalian cells. Such stimuli include pathological stresses such as oxidative damage, osmotic shock, hypoxia, and glucose deprivation, as well as physiological stimuli such as exercise, contraction, and hormones including leptin and adiponectin in skeletal muscle (Hardie, D. G., et al., 2003 FEBS Letters 546, 1113-120). AMPK is the primary regulator of the cellular response to lowered ATP levels in cultured cells. Accordingly, phosphorylation of its downstream targets results in the up-regulation of ATP-producing catabolic pathways and the downregulation of ATP-consuming processes. A number of groups have reported biochemical purification of a kinase activity (“AMPKK”) that is capable of phosphorylating Thr172 in the activation loop of AMPKα, although the identity of the kinase is currently unknown (Hawley, S. A, et al., 1996 J Biol. Chem. 271, 27879-87; Hamilton, S. R., et al., 2002 Biochem Biophys Res Commun. 293, 892-8). Though CAMKK was shown to serve as a surrogate AMPKK in vitro, a number of its biochemical and biophysical properties indicate that it is not a bona fide AMPKK in vivo (Hawley, S. A., et al., 1995 J. Biol. Chem. 270, 27186-91).

In addition to the increased clinical risks, type II diabetes may also result in a reduced quality of life for the affected individual. Because type II diabetes is a major disorder in current society, which has serious health and life quality consequences, improved methods of treatment and/or reliable diagnosis are needed and would be beneficial for patients and their families and health-care providers. By providing further insight into the biochemical pathways implicated in diabetes, identification of the AMPK kinase would advance the treatment options for diabetes.

### SUMMARY OF THE INVENTION

It has now been discovered that the LKB1 protein directly phosphorylates AMP kinase (AMPK) and activates its kinase activity. Furthermore, overexpression of wild-type LKB1 increases basal and stimulated AMPK phosphorylation and activity, whereas, a kinase-inactive LKB1 mutant acts as a dominant negative allele. Additionally, LKB1 plays a biologically significant role in this pathway since wild-type LKB1 expression surprisingly is required to prevent death of human tumor cells in response to prolonged treatment with the AMP-analogue AICAR. Therefore, LKB1 is the major AMPK kinase in mammalian cells and suggest a unexpected connection between the response of cells to metabolic stress and tumorigenesis.

In a further aspect of the invention, methods are provided for identifying compounds useful in the treatment of diabetes. The methods include determining a first amount of activity of a LKB1 polypeptide, contacting the LKB1 polypeptide with a candidate pharmacological agent, and determining the amount of activity of the contacted LKB1 polypeptide. An increase in the amount of activity in the contacted LKB1 polypeptide relative to the first amount of activity of the LKB1 polypeptide is an indication that the candidate pharmacological agent is useful in the treatment of diabetes. In a preferred embodiment, the activity of the LKB1 polypeptide is measured by phosphorylation of AMP-activated kinase.

Methods for identifying compounds useful in the treatment of diabetes are provided according to still a further aspect of the invention. The methods include providing an assay mixture comprising a LKB1 polypeptide and a STRAD polypeptide that forms a heterodimer with the LKB1 polypeptide, determining a first affinity of the dimeric interaction between LKB1 and STRAD, contacting the assay mixture with a candidate pharmacological agent, and determining a second affinity of the dimeric interaction between LKB1 and STRAD. An increase in the second affinity relative to the second affinity is an indication that the candidate pharmacological agent is useful in the treatment of diabetes. In a preferred embodiment, the affinity of the dimeric interaction between LKB1 and STRAD is measured by co-immunoprecipitating LKB1 and STRAD. In this embodiment, an increase the amount of STRAD co-immunoprecipitating with LKB1 is indicative of an increase in the affinity of the dimeric interaction.

According to another aspect of the invention, methods for identifying compounds useful in the treatment of cancer are provided. The methods include determining a first amount of activity of an AMP-activated kinase polypeptide, contacting the AMP-activated kinase polypeptide with a candidate pharmacological agent, and determining the amount of activity of the contacted AMP-activated kinase polypeptide, wherein an increase in the amount of activity in the contacted AMP-activated kinase polypeptide relative to the first amount of activity of the AMP-activated kinase polypeptide is an indication that the candidate pharmacological agent is useful in the treatment of cancer. In a preferred embodiment, the activity of the AMP-activated kinase polypeptide is measured by phosphorylation of acetyl CoA carboxylase.

According to yet another aspect of the invention, methods for preparing a diabetes drug are provided. The methods include identifying a compound that increases LKB activity and formulating the compound for administration to a subject in need of such treatment. In one embodiment, the diabetes is type II diabetes. In another embodiment, the compound that increases LKB activity is identified by the methods described herein. In a further aspect of the invention, methods for preparing a cancer drug are provided.

The methods include identifying a compound that increases AMP-activated kinase activity and formulating the compound for administration to a subject in need of such treatment. In one embodiment, the compound that increases LKB activity is identified by the methods described herein.

According to another aspect of the invention, methods for treating cancer are provided. The methods include administering to a subject having a cancer characterized by reduced or absent LKB1 activity an effective amount of a compound that increases AMP-activated protein kinase (AMPK) activity in (cells of) the subject, or a compound that increases cellular AMP levels in (cells of) the subject. In certain embodiments, the compound is an analog of adenosine monophosphate (AMP), preferably 5-aminoimidazole-4-carboxamide-1-β-D-ribofuranoside (AICAR) or an analog or derivative thereof that increases AMPK activity. In certain of these embodiments, the analog or derivative of AICAR is 5-aminoimidazole-4-carboxamide-1-β-D-ribofurano side monophosphate. In other embodiment, the analog of AMP is adenosine. In other embodiments, the compounds uncouples mitochondria, whereby cellular AMP levels are increased.

In further embodiments, the compound is metformin, rosiglitazone, leptin, adiponectin, or an analog or derivative thereof that increases AMPK activity.

In certain embodiments, the reduction of LKB1 activity is due to the mutation or deletion of the LKB1 gene.

The methods also can include subjecting the cancer (cells) of the subject to a cell death stimulus.

According to yet another aspect of the invention, methods for promoting apoptosis of cells having reduced or absent LKB1 activity are provided. The methods include contacting the cells with a compound that is an activator of AMP-activated protein kinase (AMPK), or a compound that increases cellular AMP levels. In certain embodiments, the compound is an analog of adenosine monophosphate (AMP), preferably 5-aminoimidazole-4-carboxamide-1-β-D-ribofuranoside (AICAR) or an analog or derivative thereof that increases AMPK activity. In certain of these embodiments, the analog or derivative of AICAR is 5-aminoimidazole-4-carboxamide-1-β-D-ribofurano side monophosphate. In another embodiment, the analog of AMP is adenosine. In still other embodiments, the compound uncouples mitochondria, whereby cellular AMP levels are increased.

In further embodiments, the compound is metformin, rosiglitazone, leptin, adiponectin, or an analog or derivative thereof that increases AMPK activity.

In certain embodiments, the reduction of LKB1 activity is due to the mutation or deletion of the LKB1 gene.

According to still another aspect of the invention, methods for treating a subject having or suspected of having diabetes are provided. The methods include administering to a subject in need of such treatment an effective amount of an agent that increases the activity of LKB1 in the subject, as a treatment for the diabetes. The diabetes can be type I or type II diabetes.

In certain embodiments, the agent increases the kinase activity of LKB1, and/or the amount of LKB1. In other embodiments, the agent increases the amount of STRAD. In still other embodiments, the agent increases the affinity of the dimeric interaction between LKB1 and STRAD.

Similar to the methods described above, methods for treating cancer and promoting apoptosis by inhibiting or decreasing mTOR activity or expression are provided. mTOR activity can be decreased by pharmacological inhibitors of the enzyme activity or the expression of the proteins. Preferred inhibitors are those that bind directly to the mTOR protein. Inhibitors of mTOR expression, such as siRNA or RNAi molecules, also can be used in a similar manner. Inhibitors of the activity or the expression of downstream effectors of mTOR, such as p70S6K1 or Rheb, also can be used in the treatment of the foregoing disorders.

In another aspect, the invention provides for use of the foregoing agents, compounds and molecules in the preparation of medicaments also is provided, particularly medicaments for the treatment of diabetes, obesity and reduced insulin sensitivity.

These and other aspects of the invention are described further below.

### BRIEF DESCRIPTION OF THE FIGURES

FIG. 1 shows that LKB1 prefers to phosphorylate peptide libraries with the consensus L-x-T or R-T. Wild-type (WT) or kinase-deficient (KD) LKB1 was co-expressed with STRAD in mammalian cells, purified and subjected to a panel of kinase assays using 30 μg of each peptide library. γ³²P-ATP incorporation was determined by p81 paper and scintillation counting as previously described (12). Wild-type LKB1, solid bars; Kinase-dead LKB1, open bars.

FIG. 2 shows that LKB1 phosphorylates Thr 172 of AMPKα in vitro and activates its kinase activity. FIG. 2a: Lineup of known LKB1 in vitro phosphorylation sites with sites of phosphorylation in human AMPKβ and its yeast homologue SNF1p. AMPKα1 Thr 172 (SEQ ID NO:1); SNF1p S.c. Thr 210 (SEQ ID NO:2); STRAD Thr 329 (SEQ ID NO:3); STRAD Thr 419 (SEQ ID NO:4); LKB1 auto Thr 185 (SEQ ID NO:5). FIG. 2b. HT1080 cells were transfected with wild-type or kinase dead (K78I) LKB1 with or without its coactivating protein STRAD. As indicated, LKB1 immunoprecipitations (IPs) were tested for their ability to transphosphorylate bacterial MBP-AMPKα in an in vitro kinase assay. Parallel in vitro kinase assays were performed using ³²P γ-ATP followed by autoradiography or cold ATP followed by immunoblotting for phospho-Thr172 AMPK and the indicated proteins. Wild-type LKB1 IPs were run in duplicate as shown. MBP-AMPK was also tested alone as indicated. Results are typical of 3 separate experiments. FIG. 2c: LKB1 phosphorylation of MBP-AMPK activates its kinase activity towards a peptide substrate (SAMS). LKB1 immunoprecipitates (as in FIG. 2a) were used to phosphorylate MBP-AMPK in vitro and then MBP-AMPK was removed and tested for its ability to trans-phosphorylate the SAMS peptide in the presence of ³²P γ-ATP. Results were performed in two separate experiments in triplicate. LKB1 alone was incapable of detectably phosphorylating the SAMS peptide, and equivalent levels of LKB1 and MBP-AMPK were used in each reaction (data not shown). Samples without LKB1, without SAMS peptide, or without MBP-AMPK all gave similar levels of background (data not shown).

FIG. 3 shows that LKB1-deficient mouse embryonic fibroblasts (MEFs) are defective in AMPK activation. FIG. 3a: Littermate MEFs of the indicated LKB1 genotypes were left untreated (NT) or treated with 0.1 mM H₂O₂ for 20 mins or 2 mM AICAR for 2 h. Total cell extracts were immunoblotted for phospho-Thr 172 AMPK or phospho Ser79 ACC, as well as for total AMPK and LKB1. FIG. 3b: An immortalized LKB1-deficient MEF cell line was reconstituted with human (hu) or mouse (ms) WT or KD LKB1 expressing retroviruses. The asterisk (*) represents a background band that serves as a loading control.

FIG. 4 shows that LKB1 regulates activation of AMPK in response to the AMP analogue AICAR as well as oxidative or osmotic stress in HT1080 cells. HT1080 cells stably expressing WT or KD LKB1 (as indicated in FIG. 3) were treated 0.1 mM H₂O₂ for 20 mins, 0.6M sorbitol for 30 mins, or 2 mM AICAR for 2 h. Total cell extracts were analyzed as in FIG. 3.

FIG. 5 shows that LKB1 expression protects HeLa cells from AICAR, but not UV-induced cell death. FIG. 5 shows phase contrast images of HeLa cells stably expressing vector, WT LKB1, or KD KB1 5 h after treatment with 2.5 mM AICAR. Results are representative of four independent experiments. Cell viability was determined following AICAR or UV expressed as a percentage of untreated controls. Cell viability quantified by MTT assays run in triplicate on indicated HeLa stable cell lines treated with 2.5 mM AICAR or 50 j/cm² UV for 8 h. Results are representative of two independent experiments.

FIG. 6 shows that mTOR signaling is not inhibited following energy stress in LKB1-deficient cells. FIG. 6A: Subconfluent cycling littermate-matched primary MEFs of the indicated genotype were left untreated (NT) or were treated with 2 mM AICAR for 2 h. Total cell extracts were immunoblotted for phospho-Thr172 AMPK (P-AMPK), phospho Ser79 ACC (P-ACC), phospho-Thr389 S6K1 (P-S6K1), phospho-Ser9 GSK-3b (P-GSK-3), and phospho-Ser235/236 S6 ribosomal protein (P-S6). FIG. 6B: An immortalized LKB1−/− MEF cell line was reconstituted with vector (v), wild-type (wt) or kinase-dead (kd) human LKB1-expressing retroviruses. Cells were treated as in FIG. 6A. eIF4E was used as a loading control. *represents a cross-reactive band with the anti-LKB1 antisera as characterized previously (Shaw et al. (2004) Proc. Natl. Acad. Sci. USA 101, 3329-3335). FIG. 6C: Endogenous 5′ cap complexes were isolated from LKB1 primary MEFs as in A. 10% of the total cell lysate is shown in the left panels; cap complexes purified on 7-methyl sepharose are shown in right panels. Each were immunoblotted with total 4E-BP1 and eIF4E antibodies. eIF4E is shown as a loading control. FIG. 6D: Hela cells (which are LKB1-deficient) were transiently transfected with vector, FLAG-tagged wild-type, or kinase-dead LKB1 along with an HA-tagged S6K1 or an HA-tagged 4E-BP1 construct, then treated where indicated with 2 mM AICAR for 2 h. S6K1-immunoprecipitates were immunoblotted for activating phosphorylation of S6K1 as before, as well with anti-HA to control for levels. Lysates were immunoblotted with anti-FLAG to detect levels of LKB1 and with anti-HA to detect phosphorylation induced changes in 4E-BP1 mobility on SDS-PAGE.

FIG. 7 shows that LKB1 activation of AMPK stimulates phosphorylation of tuberin in vitro and in vivo and is required for LKB1-mediated inhibition of S6K1 activity. FIG. 7A: In vitro kinase assay using FLAG-tagged tuberin as a substrate for LKB1-activated AMPK. Purified E. coli produced recombinant AMPK was activated by an in vitro kinase assay with wild-type LKB1, kinase-dead LKB1, or untransfected control immunoprecipitates then isolated and assayed for its ability to phosphorylate tuberin in vitro. FIG. 7B: Comparison of recombinant Akt-mediated tuberin phosphorylation in vitro to recombinant AMPK phosphorylation in vitro. Note that only AMPK-mediated phosphorylation induces a significant mobility shift in tuberin on SDS-PAGE. C: HeLa cells were co-transfected with FLAG-tagged tuberin, and empty vector or wild-type LKB1 and treated with 2 mM AICAR where indicated. Total cell lysates were then immunoblotted with anti-FLAG antisera to detect the introduced tuberin. Note the significant mobility shift induced by AICAR treatment of LKB1-expressing cells. AICAR treatment of cells not expressing wild-type LKB1 had no effect on tuberin mobility (data not shown). FIG. 7D: HeLa cells were transfected with HA-tagged S6K1 and FLAG-tagged wild-type LKB1 along with empty vector, or dominant negative AMPK alpha1 (GST-tagged) or dominant negative AMPK alpha2 (myc tagged). Cells were treated with 2 mM AICAR for 2 h where indicated. HA-immunoprecipitates were immunoblotted with phospho-Thr389 S6K1 antisera to detect activated S6K1 and reprobed for total HA-S6K1 levels. Lysates were immunoblotted with anti-FLAG to detect LKB1 levels and with anti-myc and anti-GST to detect expression of the dominant negative AMPK alleles.

FIG. 8 shows that LKB1- and TSC2-deficient MEFs show similar aberrant deregulation of intracellular signaling in response to specific environmental stresses. FIGS. 8A and 8B: Littermate matched LKB1- or TSC2-deficient MEFs were compared to wild-type MEFs in their response to a number of stress stimuli known to inhibit mTOR signaling (wild-type cells denoted +, −/− cells simply−). mTOR signaling was examined by immunoblotting for phospho-S6 as in FIG. 6. Activation of Akt and Erk were examined using their specific activation state phospho-specific antibodies (Ser473 for Akt, Thr202/Tyr204 for Erk). As before, eIF4E is used as a loading control. All MEFs were serum-deprived in DMEM alone for 24 h. Indicated samples were then placed in fresh media containing 10% serum, with or without 20 nm rapamycin or 2 mM AICAR, all for 90 min. Amino acid deprivation (-AA) was performed by placing the serum-starved cells into D-PBS with 10% dialyzed serum for 90 min. FIG. 8C: LKB1-deficient MEFs are hypersensitive to apoptosis induced by glucose deprivation and rapamycin rescues this apoptosis. LKB1-deficient MEFs exhibit defective AMPK activation and mTOR inhibition following glucose deprivation. Prolonged treatment in glucose-free media leads apoptosis, as indicated by the extent of caspase-mediated PARP cleavage in total cell lysates. Cells were placed in normal media, glucose free media with 10% dialyzed serum, or glucose-free media with 10% dialyzed serum containing 20 nM rapamycin for 6 or 24 h as indicated. LKB1 genotype indicated at bottom. eIF4E is used as a loading control. FIG. 8D: Cells were treated for 24 h as in FIG. 8C, and viability was quantified by MTT assays performed in triplicate on cells of the indicated genotypes. Cell death is expressed as a percentage of the untreated controls.

FIG. 9 shows that S6K1 activity is dramatically increased in GI polyps from LKB1+/− mice as compared to surrounding normal tissue. FIG. 9A: Lysates were made from polyps or adjacent normal tissue and subjected to immunoblotting with anti-phospho-S6, phospho-Erk, phospho-S6K1 (Thr389), phospho-4E-BP1 (Ser65), or total S6 as a loading control. AICAR-treated LKB1 MEFs of the indicated genotype are in the rightmost two lanes as a comparison (analogous to those in FIG. 6A). FIG. 9B: Hematoxylin and Eosin (H&E) stained section shows a polyp (upper) arising at the pyloric/duodenal junction and the adjacent normal duodenal tissue (lower). The glandular structures of polyp epithelium (pe, arrows) are embedded in the polyp stroma. The normal epithelium of the duodenum (ne) and the submucosa (sm) are indicated. FIG. 9C: Immunohistochemistry of a section adjacent to the tissue shown in FIG. 9B using anti-phospho-S6 antisera shows strong staining in the polyp epithelium and weak or absent staining in the polyp stroma and the normal duodenal tissue. FIG. 9D: Higher power view of the image shown in FIG. 9C demonstrates cytoplasmic localization phospho-S6 staining in the polyp epithelium. The scattered immunoreactive cells in the normal epithelium have a morphology suggestive of plasma cells. E: Immunohistochemistry with anti-phospho-S6 kinase antisera shows elevated cytoplasmic and nuclear staining in the polyp epithelium.

FIG. 10 shows a diagram describing upstream regulation of mTOR signaling by tumor suppressors mutated in hamartoma syndromes. In response to peptide growth factors and mitogenic stimuli, PI 3-kinase is activated resulting in the activation of the Akt serine/threonine kinase. Akt directly phosphorylates and inactivates the TSC2 tumor suppressor protein tuberin. Tuberin serves as a GTPase activating protein (GAP) for the small Ras-like GTPase Rheb. When tuberin is inactivated by Akt, Rheb-GTP levels increase, leading to increased mTOR activity via an unknown mechanism. The Pten tumor suppressor serves to biochemically antagonize PI-3 kinase activity, hence in Pten-deficient cells, Akt activity and mTOR activity are high. In response to energy stress, cells activate AMPK downstream of the LKB1 tumor suppressor. AMPK directly phosphorylates and activates tuberin, overriding the mitogenic signal from Akt. In the absence of LKB1, AMPK cannot be activated, nor mTOR inactivated via tuberin, in response to energy stress.

### BRIEF DESCRIPTION OF THE SEQUENCES

SEQ ID NO: 1
GEFLRTSCG (AMPKα1).
SEQ ID NO: 2
GNFLKTSCG (SNF1p S.c.).
SEQ ID NO: 3
SDSLTTSTP (STRAD).
SEQ ID NO: 4
IFGLVTNLE (STRAD).
SEQ ID NO: 5
GNLLLTTGG (LKB1 auto)

### Detailed Description of the Invention

AMP-activated protein kinase (AMPK, e.g., GenBank accession number AAA64745) is a highly conserved sensor of cellular energy status found in all eukaryotic cells (1). AMPK is activated by stimuli that increase the cellular AMP/ATP ratio. Essential to activation of AMPK is its phosphorylation at Thr172 by an upstream kinase (AMPKK) whose identity in mammalian cells has remained elusive (1).

The LKB1 serine/threonine kinase (e.g., GenBank accession number AAC15742) is a divergent, yet evolutionarily well-conserved kinase that most closely resembles CAMKK in its catalytic domain. LKB1 inactivation is the genetic basis of Peutz-Jeghers syndrome, a familial colorectal polyp disorder in which patients are also predisposed to early onset cancers at in other tissues (2). More recently LKB1 has also been shown to be an essential mediator of embryonic polarity in C. elegans as well as in Drosophila (9,10). STRAD (GenBank accession number BK001542), a recently identified obligate coactivator for LKB1, is the only known physiological substrate of LKB1 (11).

It now has been discovered that LKB1 specifically and directly phosphorylates AMP kinase (AMPK) and activates its kinase activity. Based on the results presented below, it appears that LKB1 is the major AMPK kinase in mammalian cells and suggest a unexpected connection between the response of cells to metabolic stress and tumorigenesis.

The data shown below suggest that compounds that specifically increase the activity (e.g., catalytic activity or the expression) of LKB1 or AMPK may be useful for treating diabetes (e.g., type II diabetes) or cancer. For example, compounds that are activators of AMPK are useful as drugs for cancer treatment and/or inducing apoptosis in cells having reduced LKB1 expression, such as adenosine, 5-aminoimidazole-4-carboxamide-1-β-D-ribofuranoside (AICAR), 5-aminoimidazole-4-carboxamide-1-β-D-ribofuranoside monophosphate, metformin, rosiglitazone, leptin, adiponectin and various analogs and derivatives thereof. Compounds that increase cellular AMP levels, such as the compounds mentioned above and compounds that uncouple mitochondria (e.g., carbonylcyanide p-trifluoromethoxyphenylhydrazone (FCCP), 2,4-dinitrophenol (DNP), carbonylcyanide m-chlorophenylhydrazone (CCCP), 5-chloro-3-tert-butyl-2′-chloro-4′-nitrosalicylanilide (S-13), and 2,6-di-t-butyl-4-(2′,2′-dicyanovinyl)phenol (SF6847)) also can be used for cancer treatment and/or inducing apoptosis in cells having reduced LKB1 expression. Agents that increase the expression of LKB1 or AMPK (either of the endogenous gene or by introducing one or more copies of the gene) also can be used for these purposes. In addition, compounds that increase the dimerization of LKB1 and STRAD also are useful for the same purposes. Accordingly, the invention also provides methods for identifying agents useful in treating these disorders by increasing the activity (e.g., kinase activity or dimerization of LKB1 and STRAD) or expression of LKB1 or AMPK.

Likewise, the data shown in the Examples below suggest that compounds that specifically inhibit the activity (e.g., catalytic activity or the expression) of mTOR (mammalian target of rapamycin) may be useful for treating cancer in which LKB1 is deficient, such as hamartomas typical of Peutz-Jeghers syndrome and lung adenocarcinomas. For example, compounds that are inhibitors of mTOR are useful as drugs for cancer treatment and/or inducing apoptosis in cells having reduced LKB1 expression. Therefore, the invention includes the use of inhibitors of mTOR activity or expression for cancer treatment as is described herein for compounds that specifically increase the activity (e.g., catalytic activity or the expression) of LKB1 or AMPK.

Any method for decreasing mTOR activity or expression will be useful in the treatment of the aforementioned disorders. mTOR activity can be decreased by pharmacological inhibitors of the enzyme activity or the expression of the proteins. Preferred inhibitors are those that bind directly to the mTOR protein. Inhibitors of the activity or the expression of downstream effectors of mTOR, such as p70S6K1 or Rheb, also can be used in the treatment of the foregoing disorders.

In accordance with the invention, known inhibitors of mTOR that can be used include rapamycin (sirolimus), and rapamycin analogs and derivatives such as everolimus (RAD-001), rapamycin 42-ester with 3-hydroxy-2-(hydroxymethyl)-2-methylpropionic acid (CCI-779) (see also Dutcher, Curr. Oncol. Rep. 6(2):111-115, 2004; Huang and Houghton, Curr. Opin. Investig. Drugs 3(2):295-304, 2002; and U.S. Pat. Nos. 5,362,718; 5,780,462; 5,922,730; 5,955,457; 6,015,809; 6,117,863; 6,399,625; 6,399,626; 6,432,973; 6,440,991; 6,677,357; and 6,680,330). Known inhibitors of p70S6K1 include N-[2-(p-bromocinnamylamino)ethyl]-5-isoquinoline sulfonamide (H89), 1-(5-isoquinolinesulfonyl)-1H-hexahydro-1,4-diazepine (HA-1077, fasudil). Known inhibitors of Rheb include farnesyl transferase inhibitors, e.g., FTI 277, Zarnestra® (tipifarnib, R115777), RPR-130401, lonafarnib (SCH66336).

Agents that decrease the expression of mTOR (e.g., by decreasing expression of the endogenous gene) also can be used for these purposes. Such agents include antisense nucleic acid molecules and siRNA or RNAi molecules.

One set of embodiments of the aforementioned compositions and methods include the use of antisense molecules or nucleic acid molecules that reduce expression of genes via RNA interference (RNAi or siRNA). One example of the use of antisense, RNAi or siRNA in the methods of the invention is their use to decrease the level of expression of one or more ceramide biosynthetic pathway enzymes. The antisense oligonucleotides, RNAi, or siRNA nucleic acid molecules used for this purpose may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods, which may be carried out manually or by an automated synthesizer. They also may be produced recombinantly by vectors.

In some embodiments of the invention, the antisense or siRNA oligonucleotides also may include “modified” oligonucleotides. That is, the oligonucleotides may be modified in a number of ways, which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars that are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acid molecules encoding proteins of the invention, together with pharmaceutically acceptable carriers.

The use of RNA interference or “RNAi” involves the use of double-stranded RNA (dsRNA) to block gene expression. (see: Sui, G, et al, Proc Nall. Acad. Sci. U.S.A. 99:5515-5520, 2002). Methods of applying RNAi strategies in embodiments of the invention would be understood by one of ordinary skill in the art.

Methods in which small interfering RNA (siRNA) molecules are used to reduce the expression of mTOR may be used. In one aspect, a cell is contacted with a siRNA molecule to produce RNA interference (RNAi) that reduces expression of one or more of the aforementioned genes. The siRNA molecule is directed against nucleic acids coding for the relevant polypeptide (e.g. RNA transcripts including untranslated and translated regions). Well known methods such as Western blotting can be used for determining the level of protein expression and Northern blotting or RT-PCR can be used for determining the level of mRNA transcript of the targeted gene.

As used herein, a “siRNA molecule” is a double stranded RNA molecule (dsRNA) consisting of a sense and an antisense strand. The antisense strand of the siRNA molecule is a complement of the sense strand (Tuschl, T. et al., 1999, Genes & Dev., 13:3191-3197; Elbashir, S. M. et al., 2001, EMBO J., 20:6877-6888; incorporated herein by reference). In one embodiment the last nucleotide at the 3′ end of the antisense strand may be any nucleotide and is not required to be complementary to the region of the target gene. The siRNA molecule preferably is 19-23 nucleotides in length and may form a hairpin structure. In one preferred embodiment the siRNA molecule includes a two nucleotide 3′ overhang on the sense strand. In a second preferred embodiment the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first nucleotide of the siRNA molecule is a purine.

The siRNA molecules can be plasmid-based. In a preferred method, a polypeptide encoding sequence of one of the aforementioned genes is amplified using the well known technique of polymerase chain reaction (PCR). The use of the entire polypeptide encoding sequence is not necessary; as is well known in the art, a portion of the polypeptide encoding sequence is sufficient for RNA interference. The PCR fragment is inserted into a vector using routine techniques well known to those of skill in the art. Combinations of the foregoing can be expressed from a single vector or from multiple vectors introduced into cells.

In one aspect of the invention, a mammalian vector comprising any of the nucleotide coding sequences of the invention is provided. The mammalian vectors include but are not limited to the pSUPER RNAi vectors (Brummelkamp, T. R. et al., 2002, Science, 296:550-553, incorporated herein by reference). In one embodiment, a nucleotide coding sequence can be inserted into the mammalian vector using restriction sites, creating a stem-loop structure. In a second embodiment, the mammalian vector may comprise the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. The antisense strand of the siRNA molecule hybridizes to the corresponding region of the mRNA of the target gene.

Preferred systems for mRNA expression in mammalian cells are those such as pSUPER RNAi system as described in Brummelkamp et al. (2002, Science, 296:550-553). Other examples include but are not limited to pSUPER.neo, pSUPER.neo+gfp, pSUPER.puro, BLOCK-iT T7-TOPO linker, pcDNA1.2/V5-GW/lacZ, pENTR/U6, pLenti6-GW/U6-laminshrna, and pLenti6/BLOCK-iT-DEST. These vectors are available from suppliers such as Invitrogen, and one of skill in the art would be able to obtain and use them.

As used herein, the term “aberrantly” means abnormally, and may include increased expression or functional activity and/or decreased expression or functional activity.

As used herein, a subject is preferably a human, non-human primate, cow, horse, pig, sheep, goat, dog, cat, or rodent. In all embodiments, human subjects are preferred. In some embodiments, the subject is suspected of having a disorder associated with insufficient or decreased LKB1 or AMPK activity such as diabetes or cancer.

Methods for identifying subjects suspected of having a LKB1- or AMPK-associated disorder may include but are not limited to: physical examination, subject's family medical history, subject's medical history, blood tests, visual exam, mean body mass assessment, and/or weight assessment. Diagnostic methods for LKB1- or AMPK-associated disorders are well-known to those of skill in the medical arts, although not necessarily with respect to LKB1 or AMPK activity.

As used herein, a biological sample includes, but is not limited to: tissue, cells, or body fluid (e.g. blood or lymph node fluid). The fluid sample may include cells and/or fluid. The tissue and cells may be obtained from a subject or may be grown in culture (e.g. from a cell line). The type of biological sample may include, but is not limited to: colon tissue (including polyps), skeletal muscle, brain, and/or adipose tissue, which also may be referred to herein as “fat.” In some embodiments of the invention, the biological sample is a control sample.

As used herein a “control” may be a predetermined value, which can take a variety of forms. It can be a single cut-off value, such as a median or mean. It can be established based upon comparative groups, such as in groups having normal amounts of circulating insulin and groups having abnormal amounts of circulating insulin, or individuals with colorectal polyps and individuals without colorectal polyps. Another example of comparative groups would be groups having a particular disease, condition or symptoms and groups without the disease, condition or symptoms. Another comparative group would be a group with a family history of a condition and a group without such a family history. The predetermined value can be arranged, for example, where a tested population is divided equally (or unequally) into groups, such as a low-risk group, a medium-risk group and a high-risk group or into quandrants or quintiles.

The predetermined value, of course, will depend upon the particular population selected. For example, an apparently healthy population will have a different ‘normal’ range than will a population which is known to have a condition related to aberrant LKB1 or AMPK molecule expression or activity. Accordingly, the predetermined value selected may take into account the category in which an individual falls. Appropriate ranges and categories can be selected with no more than routine experimentation by those of ordinary skill in the art. By abnormally high it is meant high relative to a selected control. Typically the control will be based on apparently healthy normal individuals in an appropriate age bracket.

In some embodiments, a control sample is from a cell, tissue, or subject that does not have a disorder associated with insufficient LKB1 or AMPK activity. In other embodiments the control sample is a sample that is untreated with a candidate agent. For example, an effect of a candidate agent may be determined by determining the catalytic activity of a LKB1 or AMPK polypeptide in advance of contacting the LKB1 or AMPK polypeptide with the agent, and again after contacting the LKB1 or AMPK polypeptide with the agent, in which case, the initial level of catalytic activity determined may serve as a control level against with the post-contact level of catalytic activity may be compared. In such assays, the source of the LKB1 or AMPK polypeptide may be a biological sample known to be free of a LKB1- or AMPK-associated disorder or may be a sample from a cell or tissue with a known LKB1- or AMPK-associated disorder, and in each case the before-contact determination of catalytic activity may be the control for the after-contact determination of catalytic activity.

The phrase “suspected of having a LKB1- or AMPK-associated disorder” and the like as used herein means a tissue or tissue sample believed by one of ordinary skill in the art to contain aberrant levels or activity of LKB1 or AMPK nucleic acid molecules and/or the polypeptides they encode. Examples of methods for obtaining the sample from the biopsy include aspiration, gross apportioning of a mass, microdissection, laser-based microdissection, or other art-known cell-separation methods.

Because of the variability of the cell types in diseased-tissue biopsy material, and the variability in sensitivity of the diagnostic methods used, the sample size required for analysis may range from 1, 10, 50, 100, 200, 300, 500, 1000, 5000, 10,000, to 50,000 or more cells. The appropriate sample size may be determined based on the cellular composition and condition of the biopsy and the standard preparative steps for this determination and subsequent isolation of the nucleic acid for use in the invention are well known to one of ordinary skill in the art. An example of this, although not intended to be limiting, is that in some instances a sample from the biopsy may be sufficient for assessment of RNA expression without amplification, but in other instances the lack of suitable cells in a small biopsy region may require use of RNA conversion and/or amplification methods or other methods to enhance resolution of the nucleic acid molecules. Such methods, which allow use of limited biopsy materials, are well known to those of ordinary skill in the art and include, but are not limited to: direct RNA amplification, reverse transcription of RNA to cDNA, real-time RT-PCR, amplification of cDNA, or the generation of radiolabeled nucleic acids.

The surprising discovery that LKB1 phosphorylates AMPK, which modulates its activity, and is related to various disorders provides for novel methods of treatment of such disorders in which aberrant LKB1 or AMPK activity is involved. In particular, methods for treating LKB1- or AMPK-associated disorders are provided by the invention, in which LKB1 or AMPK activity is increased, which increases phosphorylation of the respective substrates of these kinases. Activity of LKB1 and AMPK, as used herein in these contexts, means kinase activity (for LKB1 and AMPK) and/or dimerization (for LKB1).

Any method for increasing LKB1 or AMPK activity will be useful in the treatment of disorders. LKB1 or AMPK activity can be increased by pharmacological activators of the enzyme activity or its expression.

Treatment for a LKB1- or AMPK-associated disorder may include, but is not limited to: surgical intervention, dietetic therapy, and pharmaceutical therapy. In some embodiments, treatment may include administration of a pharmaceutical agent that increases LKB1 or AMPK activity. The inhibitors of LKB1 or AMPK activity can be administered in conjunction with other pharmaceutical agents known for treatment of such disorders. For example, in the treatement of type II diabetes, other therapeutics such as insulin sensitizers, insulin secretagogues, insulin, and the like can be administered in conjunction (simultaneously or sequentially) with therapeutics that increase LKB1 or AMPK activity or expression Likewise, for treatment of cancers having low LKB1 activity, therapeutics that increase or stimulate LKB1 or AMPK activity or expression can be administered in conjunction with other cancer therapeutics or in conjunction with surgery.

The amino acid sequences identified herein as LKB1 or AMPK polypeptides, and the nucleotide sequences encoding them, are sequences deposited in databases such as GenBank. The use of these known LKB1 or AMPK sequences in pharmaceutical screening assays, determination of pharmaceutical agents, and diagnostic assays for LKB1- or AMPK-associated disorders as described herein is novel. Homologs, alleles, and other variants of the LKB1 or AMPK nucleic acid sequences and polypeptides sequences can also be used, as appropriate, as will be known to one of ordinary skill in the art. In general, homologs, alleles and other variants typically will share at least 90% nucleotide identity and/or at least 95% amino acid identity to the sequences of a LKB1 or AMPK nucleic acid and polypeptide, respectively, in some instances will share at least 95% nucleotide identity and/or at least 97% amino acid identity, and in other instances will share at least 97% nucleotide identity and/or at least 99% amino acid identity. The homology can be calculated using various, publicly available software tools developed by NCBI (Bethesda, Md.) that can be obtained through the Internet or using a variety of commercially available softward packages. Exemplary tools include the BLAST system available from the website of the National Center for Biotechnology Information (NCBI) at the National Institutes of Health.

The identification herein of LKB1 or AMPK polypeptides as involved in physiological disorders also permits the artisan to diagnose a disorder characterized by expression of LKB1 or AMPK polypeptides, and characterized preferably by an alteration in functional activity of the LKB1 or AMPK polypeptides.

Determination of the catalytic activity of LKB1 or AMPK polypeptides for diagnostic, prognostic, and therapeutic purposes is an aspect of the invention. The catalytic activity of a LKB1 or AMPK polypeptide may be determined and candidate pharmaceutical agents can be tested for their ability to modify (decrease or increase) the LKB1 or AMPK catalytic activity. The determination that a compound modifies the LKB1 or AMPK catalytic activity indicates that the compound may be useful as an agent to treat LKB1 or AMPK -associated disorders, such as type II diabetes or cancer. For example, a LKB1 or AMPK polypeptide may be contacted with a substrate of the polypeptide and the catalytic activity of the LKB1 or AMPK monitored and determined, then the LKB1 or AMPK polypeptide may be contacted with a candidate agent and the polypeptide's catalytic activity determined upon contact with the substrate. Such assays may be done in vitro and may also be useful to monitor effects of in vivo administration of catalytic activity modulators in cells or animals, including humans.

The invention also involves the use of agents such as polypeptides that bind to LKB1 or AMPK polypeptides or substrates of such polypeptides. Such binding agents can be used, for example, in screening assays to detect the presence or absence of LKB1 or AMPK polypeptides or their substrates and in purification protocols to isolate LKB1 or AMPK, their substrates or complexes of LKB1 or AMPK polypeptides and their substrates.

The invention, therefore, embraces peptide binding agents which, for example, can be antibodies or fragments of antibodies having the ability to selectively bind to LKB1 or AMPK polypeptides or their substrates. Antibodies include polyclonal and monoclonal antibodies, prepared according to conventional methodology. As used herein, LKB1 or AMPK antibodies, are antibodies that specifically bind to LKB1 or AMPK polypeptides, respectively.

Significantly, as is well known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to LKB1 or AMPK polypeptides, their substrates and complexes of both LKB1 or AMPK polypeptides and their substrates. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the LKB1 or AMPK polypeptide. This process can be repeated through several cycles of reselection of phage that bind to the LKB1 or AMPK polypeptide. Repeated rounds lead to enrichment of phage bearing particular sequences. DNA sequence analysis can be conducted to identify the sequences of the expressed polypeptides. The minimal linear portion of the sequence that binds to the LKB1 or AMPK polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the LKB1 or AMPK polypeptides.

The invention also relates in part to methods of treating disorders associated with insufficient or otherwise aberrant LKB1 or AMPK activity, such as: diabetes (particularly type II) and cancer. An “effective amount” of a drug therapy is that amount of an agent that increases LKB1 or AMPK activity that alone, or together with further doses, produces the desired response, e.g. reduction of symptoms of type II diabetes, slowing or reversing the progression of cancer, or increasing apoptosis of cancer cells.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the agent that increases LKB1 or AMPK activity (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an agent that increases LKB1 or AMPK activity for producing the desired response in a unit of weight or volume suitable for administration to a patient.

The doses of an agent that increases LKB1 or AMPK activity administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Various modes of administration will be known to one of ordinary skill in the art which effectively deliver the agent that increases LKB1 or AMPK activity to a desired tissue, cell or bodily fluid. Administration includes: topical, intravenous, oral, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of agent that increases LKB1 or AMPK activity will be known to one of ordinary skill in the art, in which the dose amount, schedule of administration, sites of administration, mode of administration (e.g., intra-organ) and the like vary from those presented herein.

Administration of agents that increase LKB1 or AMPK activity to mammals other than humans, e.g. for testing purposes or veterinary therapeutic purposes, is carried out under substantially the same conditions as described above. It will be understood by one of ordinary skill in the art that this invention is applicable to both human and animal diseases that can be treated by an agent that increases LKB1 or AMPK activity. Thus this invention is intended to be used in husbandry and veterinary medicine as well as in human therapeutics.

In general, for treatments of disorders, doses of agents that increases LKB1 or AMPK activity are formulated and administered in doses between 0.2 mg to 5000 mg of the agent that increases LKB1 or AMPK. Preferably, an effective amount will be in the range from about 0.5 mg to 500 mg of the agent that increases LKB1 or AMPK activity, according to any standard procedure in the art. Administration of agents that increases LKB1 or AMPK activity compositions to mammals other than humans, e.g. for testing purposes or veterinary therapeutic purposes, is carried out under substantially the same conditions as described above. A therapeutically effective amount typically varies from 0.01 ng/kg to about 1000 μg/kg, preferably from about 0.1 ng/kg to about 200 μg/kg and most preferably from about 0.2 ng/kg to about 20 μg/kg, in one or more dose administrations daily, for one or more days.

The pharmaceutical preparations of the invention may be administered alone or in conjunction with standard treatment(s) of disorders associated with alterations in LKB1 or AMPK activity, such as diabetes or cancer. For example, treatment for type II diabetes with a pharmaceutical agent of the invention, may be undertaken in parallel with treatments for diabetes that is known and practiced in the art. For example, such treatments may include, but are not limited to administration of metformin, pioglitazone, and/or rosiglitazone. Other known treatments for type II diabetes include pharmaceutical agents that increases insulin release, which may include, but are not limited to sulfonylureas, nateglinide and repaglinide. In some treatment methods, sulfonylureas include, but are not limited to glibenclamide (glyburide), gliclazide and glimepiride. In some embodiments of the invention, insulin may be administered to the subject, in conjunction with the treatment methods of the invention.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. Preferred components of the composition are described above in conjunction with the description of the agent that increases LKB1 or AMPK activity of the invention.

An agent that increases LKB1 or AMPK activity composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the agent that increases LKB1 or AMPK activity, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise an agent that increases LKB1 or AMPK activity. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A long-term sustained release implant also may be used for administration of the pharmaceutical agent composition. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by insufficient increases LKB1 or AMPK activity by placing the implant near portions of a subject affected by such activity, thereby effecting localized, high doses of the compounds of the invention.

The invention also relates in part to assays used to determine the catalytic activity of a LKB1 or AMPK polypeptide, and/or the affinity of interactions between proteins that influence such activity, e.g., the affinity of dimerization between LKB1 and STRAD. The LKB1 or AMPK polypeptide may be attached to a surface and then contacted with a substrate molecule and the level of catalytic activity of the LKB1 or AMPK polypeptide or fragment thereof can be monitored and quantitated using standard methods. The aforementioned assays are not intended to be limiting. Assays for catalytic activity may also be done with the components in solution, using various art-recognized detection methods, and/or other kinase assay methods known to one of ordinary skill in the art, some of which are described herein below. Typically these will be kinase assays as are well known in the art; certain examples are provided in the Examples below.

The invention further provides efficient methods of identifying pharmacological agents or lead compounds for agents useful for increasing LKB1 or AMPK kinase activity. Generally, the screening methods involve assaying for compounds that modulate (e.g., enhance) phosphorylation of a substrate, or that modulate (e.g., enhance) affinity of molecular interactions, such as LKB1-STRAD heterodimer formation. Such methods are adaptable to automated, high throughput screening of compounds.

A wide variety of assays for pharmacological agents are provided, including labeled in vitro kinase phosphorylation assays, cell-based phosphorylation assays, assays for determining affinity of interacting proteins (e.g., immunoprecipitations, two-hybrid assays) etc. For example, in vitro kinase phosphorylation assays are used to rapidly examine the effect of candidate pharmacological agents on the phosphorylation of a substrate by, for example, LKB1 or AMPK or a fragment thereof. Assays of intermolecular interaction, such as LKB1-STRAD heterodimer formation, also are known in the art. Compounds that increase such interactions can be determined using these assays; examples of compounds that increase protein interactions include FK506 and cyclosporin, which form bridges between proteins. Therefore, compounds that modulate LKB1-STRAD heterodimer formation can be identified for example by screening for compounds that increase the amount of STRAD co-immunoprecipitating with LKB1, for example using a cell based assay. Other well known assays of dimer formation, such as yeast two-hybrid transcription assays also can be employed to screen for compounds that modulate LKB1-STRAD heterodimer formation.

The candidate pharmacological agents can be derived from, for example, combinatorial peptide or small molecule libraries. Convenient reagents for such assays are known in the art.

In general, substrates used in the assay methods of the invention are added to an assay mixture as an isolated molecule. For use with LKB1, a preferred substrate is AMPK, although STRAD phosphorylation or LKB1 autophosphorylation also can be detected. For AMPK, a preferred substrate is acetyl CoA carboxylase. Still other substrates for the kinases will be known to one of ordinary skill in the art. The assay mixture can include detectable phosphate compounds (e.g. ³²P or ³³P), so that protein substrates phosphorylated by LKB1 or AMPK are readily detectable. Alternatively, LKB1 or AMPK activity on a substrate can be measured using other detectable means such as antibody capture of specific phosphorylated polypeptides, chromatographic means, etc. As noted above, the affinity of LKB1-STRAD heterodimers can be assayed by immunoprecipitations or by another assay of affinity.

A typical assay mixture for measuring kinase activity includes a peptide having a phosphorylation site motif and a candidate pharmacological agent. A typical assay mixture for measuring intermolecular interactions includes the molecules expected to interact, e.g., LKB1 and STRAD as described herein. Fragments of these molecules that participate in the dimerization (e.g., dimerization domains) can also be employed, optionally fused to, or labeled with, moieties that provide detection functionality. Examples of this are fusion proteins containing LKB1 or STRAD fused to transcription activator domains, domains with enzymatic activity, or directly detectable domains (e.g., fluorescent proteins, protein tags recognized by specific antibodies).

Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides (e.g., kinase sites), and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid (i.e., aptamer), the agent typically is a DNA or RNA molecule, although modified nucleic acids having non-natural bonds or subunits are also contemplated. LKB1 or AMPK activators, or modulators of LKB1-STRAD interaction, also can be designed using rational structure-based methods such as the methods described in PCT/US98/10876 and references described therein.

Candidate agents are obtained from a wide variety of sources including libraries of synthetic or natural compounds. For example, numerous means are available for random and directed synthesis of a wide variety of organic compounds and biomolecules, including expression of randomized oligonucleotides, random or non-random peptide libraries, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as nuclease inhibitors, antimicrobial agents, and the like may also be used.

The mixture of the foregoing assay materials is incubated under conditions whereby, but for the presence of the candidate pharmacological agent, LKB1 or AMPK phosphorylates a polypeptide at a certain level (i.e., control level). For affinity assays, the foregoing assay materials are incubated under conditions whereby, but for the presence of the candidate pharmacological agent, LKB1 dimerizes with STRAD polypeptide at a certain level (i.e., control level). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the presence or absence of phosphorylation of a substrate, binding of a substrate, or dimerization is detected by any convenient method available to the user. For cell free binding type assays, a separation step may be used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific binding or interaction such as salts, buffer, detergent, non-specific protein, etc. Where the solid substrate is a magnetic bead, the beads may be washed one or more times with a washing solution and isolated using a magnet.

Detection may be effected using any convenient method, some of which are described in greater detail in the Examples below. For example, phosphorylation produces a directly or indirectly detectable product, e.g., phosphorylated substrate. In other assays, one of the components usually comprises, or is coupled to, a detectable label. A wide variety of labels can be used, such as those that provide direct detection (e.g., radioactivity, luminescence, fluorescence, optical or electron density, etc). or indirect detection (e.g., epitope tag such as the FLAG, V5 or myc epitopes, an enzyme tag such as horseradish peroxidase or luciferase, a transcription product, etc.). The label may be bound to a substrate or inhibitor as described elsewhere herein, to the proteins employed in the assays, or to the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the present invention includes automated drug screening assays for identifying compositions having the ability to increase phosphorylation of a substrate directly or indirectly. The automated methods preferably are carried out in an apparatus which is capable of delivering a reagent solution to a plurality of predetermined compartments of a vessel and measuring the change in a detectable molecule in the predetermined compartments. Exemplary methods include the following steps. First, a divided vessel is provided that has one or more compartments which contain a substrate which, when exposed to LKB1 or AMPK, has a detectable change. The LKB1 or AMPK can be in a cell in the compartment, in solution, or immobilized within the compartment. Next, one or more predetermined compartments are aligned with a predetermined position (e.g., aligned with a fluid outlet of an automatic pipette) and an aliquot of a solution containing a compound or mixture of compounds being tested for its ability to increase LKB1 or AMPK kinase activity is delivered to the predetermined compartment(s) with an automatic pipette. The substrate also can be added with the compounds or following the addition of the compounds. Finally, detectable signal; emitted by the substrate is measured for a predetermined amount of time, preferably by aligning said cell-containing compartment with a detector. Preferably, the signal also measured prior to adding the compounds to the compartments, to establish e.g., background and/or baseline values. For competition assays, the compounds can be added with or after addition of a substrate or inhibitor to the LKB1 or AMPK polypeptide-containing compartments. One of ordinary skill in the art can readily determine the appropriate order of addition of the assay components for particular assays.

At a suitable time after addition of the reaction components, the plate is moved, if necessary, so that assay wells are positioned for measurement of signal. Because a change in the signal may begin within the first few seconds after addition of test compounds, it is desirable to align the assay well with the signal detector as quickly as possible, with times of about two seconds or less being desirable. In preferred embodiments of the invention, where the apparatus is configured for detection through the bottom of the well(s) and compounds are added from above the well(s), readings may be taken substantially continuously, since the plate does not need to be moved for addition of reagent. The well and detector device should remain aligned for a predetermined period of time suitable to measure and record the change in signal.

The apparatus of the present invention is programmable to begin the steps of an assay sequence in a predetermined first well (or rows or columns of wells) and proceed sequentially down the columns and across the rows of the plate in a predetermined route through well number n. It is preferred that the data from replicate wells treated with the same compound are collected and recorded (e.g., stored in the memory of a computer) for calculation of signal.

To accomplish rapid compound addition and rapid reading of the response, the detector can be modified by fitting an automatic pipetter and developing a software program to accomplish precise computer control over both the detector and the automatic pipetter. By integrating the combination of the fluorometer and the automatic pipetter and using a microcomputer to control the commands to the detector and automatic pipetter, the delay time between reagent addition and detector reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple signal measurements, reliability of the assays as well as the number of assays that can be performed per day are advantageously increased.

Similar assays can be used to identify compounds that decrease LKB1 or AMPK activity, which may be useful as controls or in preparing animal models of disease. Likewise, similar assays can be used to identify compounds that modulate LKB1-STRAD dimerization.

Activators of LKB1 or AMPK polypeptide activity (e.g., phosphorylation activity, dimerization activity) identified by the methods described herein are useful to treat diseases or conditions that result from reduced or insufficient LKB1 or AMPK polypeptide activity, including diabetes (particularly type II diabetes), cancer, etc. For treatment of such conditions, an effective amount of a LKB1 or AMPK polypeptide activator is administered to a subject.

The invention includes kits for assaying the activity level of a LKB1 or AMPK polypeptide (e.g., phosphorylation activity, dimerization activity) for determining whether test compounds modulate (preferably increase) the LKB1 or AMPK polypeptide activity. One example of such a kit of the invention is a kit that provides components necessary to determine the activity level of a LKB1 or AMPK polypeptide of the invention using a kinase assay. The components can include an appropriate substrate molecule as well as necessary cofactors and other components (e.g., buffers, radioactive molecules). Another example is a kit that provides components necessary to determine the dimerization level of LKB1 and STRAD polypeptides using an assay of intermolecular proximity, such as a dimerization assay, or a two hybrid assay.

Another example of a kit of the invention, is a kit that provides components necessary to determine the level of expression of a LKB1 or AMPK nucleic acid molecule of the invention. Such components may include, primers useful for amplification of a LKB1 or AMPK nucleic acid molecule and/or other chemicals for PCR amplification.

The foregoing kits can include instructions or other printed material on how to use the various components of the kits for diagnostic purposes or for compound screening purposes.

The invention also includes methods to monitor the onset, progression, or regression of a disorder associated with insufficient LKB1 or AMPK activity in a subject by, for example, obtaining samples at sequential times from a subject and assaying such samples for the level of expression of LKB1 or AMPK nucleic acid molecules, the level of expression of LKB1 or AMPK polypeptide molecules, and/or the level of activity of a LKB1 or AMPK polypeptide (including phosphorylation activity and dimerization activity). A subject may be suspected of having a disorder associated with insufficient LKB1 or AMPK activity or may be believed not to have such a disorder and in the latter case, the sample expression or activity level may serve as a control for comparison with subsequent samples.

Onset of a condition is the initiation of the changes associated with the condition in a subject. Such changes may be evidenced by physiological symptoms, or may be clinically asymptomatic. For example, the onset of a disorder associated with insufficient LKB1 or AMPK activity may be followed by a period during which there may be physiological changes in the subject, even though clinical symptoms may not be evident at that time. The progression of a condition follows onset and is the advancement of the physiological elements of the condition, which may or may not be marked by an increase in clinical symptoms. Onset and progression are similar in that both represent an increase in the characteristics of a disorder (e.g. expression or activity of LKB1 or AMPK molecules), in a cell or subject, onset represents the beginning of this disorder and progression represents the worsening of a preexisting condition. In contrast to onset and progression, regression of a condition is a decrease in physiological characteristics of the condition, perhaps with a parallel reduction in symptoms, and may result from a treatment or may be a natural reversal in the condition.

A marker for disorders associated with insufficient LKB1 or AMPK activity may be the level or amount of catalytic activity of a LKB1 or AMPK polypeptide, the level or amount of specific phosphorylation of a LKB1 or AMPK substrate, or the level of expression of a LKB1 or AMPK nucleic acid or polypeptide.

### EXAMPLES

### Example 1

### Summary

AMP-activated protein kinase (AMPK) is a highly conserved sensor of cellular energy status found in all eukaryotic cells (1). AMPK is activated by stimuli that increase the cellular AMP/ATP ratio. Essential to activation of AMPK is its phosphorylation at Thr172 by an upstream kinase (AMPKK) whose identity in mammalian cells has remained elusive (1). Here we present biochemical and genetic evidence indicating that the LKB1 serine/threonine kinase—the gene inactivated in the Peutz-Jeghers familial cancer syndrome (2)—is the dominant regulator of AMPK activation in several mammalian cell types. We show that LKB1 directly phosphorylates Thr172 of AMPKα in vitro and activates its kinase activity. Lkb1-deficient murine embryonic fibroblasts (MEFs) show nearly complete loss of Thr172 phosphorylation and downstream AMPK signaling in response to a variety of stimuli that activate AMPK. Reintroduction of wild-type but not kinase dead LKB1 into these cells restores AMPK activity. Furthermore, in a number of cell types overexpression of wild-type LKB1 increases basal and stimulated AMPK phosphorylation and activity, whereas, a kinase-inactive LKB1 mutant acts as a dominant negative allele. Finally, we show that LKB1 plays a biologically significant role in this pathway since wild-type LKB1 expression is required to prevent death of human tumor cells in response to prolonged treatment with the AMP-analogue AICAR. These results indicate that LKB1 may be the major AMPKK in mammalian cells and suggest a unexpected connection between the response of cells to metabolic stress and tumorigenesis. The role of LKB1/AMPK in survival of some tumor cells may provide novel opportunities for cancer therapeutics.

### Methods

Materials

HT1080, LLC-PK1, and HeLa cells were all purchased from American Type Tissue Collection (ATCC). Mouse embryonic fibroblasts (MEFs) were derived from 13.5 postcoitum embryos as previously described (15). Lkb1−/− MEFs were produced by in vitro excision of the Lkb1 lox allele as previously described (15). P-AMPK, AMPK, and P-ACC antibodies were from Cell Signaling. SAMS peptide was from Upstate Biotechnology. MBP-AMPKa (1-312) bacterial fusion protein was a generous gift from L. Witters (Dartmouth Medical School). LKB1 antibody (1G) was previously described (15). Flag-tagged human LKB1 was generated by subcloning the human LKB1 cDNA into a N-terminal tagged pcDNA3 vector. Human and mouse LKB1 retroviral constructs were generated by PCR and subcloning into pBABE-puro. Point mutations were generated using Quickchange mutatagenesis (Stratagene). STRAD was PCR amplified from a human EST (Research Genetics) and subcloned into pcDNA4 HisMax (Invitrogen). All constructs were fully sequenced to verify their integrity. AICAR (5-aminoimidazole-4-carboxamide 1-β-D-ribofuranoside) from Toronto Research Chemicals. Sorbitol, H₂O₂, MTT all from Sigma. UV light was delivered using a Stratalinker 2400 (Stratagene).

Kinase Assays and Cellular Analysis: AMPK kinase activity was measured using the SAMS peptide as previously described (7). LKB1 phosphorylation of AMPK was carried out in kinase buffer (50 mM Tris pH 7.5, 10 mM MgCl₂, 1 mM DTT, 100 μM ATP) for 20 mins at 30° C. For immunopreciptations of active LKB1 kinase, Flag-tagged LKB1 was co-transfected with an equimolar amount of STRAD expression plasmid into HT1080 cells and immunoprecipitated using M2-agarose (Sigma) 24 h post-transfection in NP40 lysis buffer26. Before kinase assays, immunoprecipitated LKB1 was washed 3× in lysis buffer then 2× in kinase buffer. Soluble MBP-AMPK was added at 5 μg per kinase reaction. The LKB1 peptide library screen was performed as previously described₁₂. Peptide libraries were fixed at indicated positions and degenerate for all 20 amino acids minus cysteine, threonine, and serine at all other positions indicated with an x; with at least four degenerate flanking positions on either side of all fixed sequences (e.g. LxT library is composed of x-x-x-x-Leu-x-Thr-x-x-x peptides with a fixed lysine tail (K-K-K). Total cell extracts and immunoblotting were as previously described (27). Amphotropic and ecotropic retroviral infections and subsequent selections were as previously described (28). For the cell survival assays, cells were plated in triplicate for each condition in 48 well plates. MTT assays were performed according to manufacturer's suggestions (Sigma).

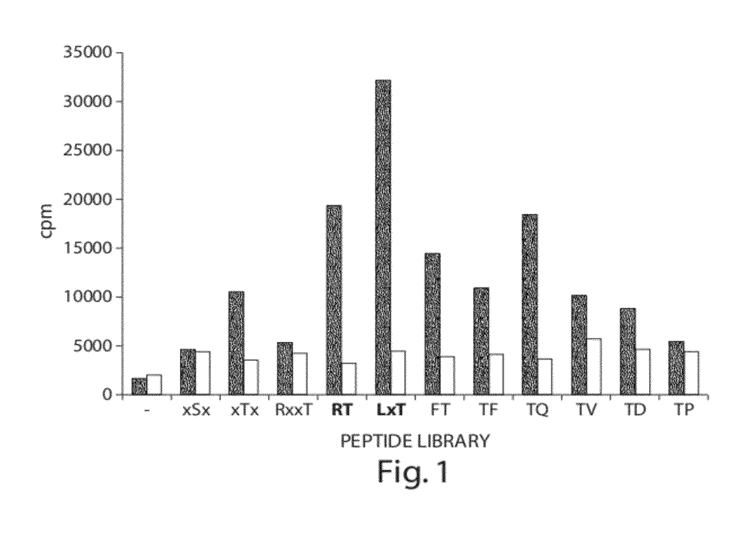

We set out to identify the optimal substrate motif for LKB1 in an attempt to identify other substrates. To examine the substrate specificity of LKB1, we co-expressed it in mammalian cells with its co-activator STRAD, and then tested the ability of purified LKB1 immunoprecipitates to phosphorylate various degenerate peptide libraries (12). First, we found that LKB1 will only phosphorylate libraries with threonine as the phosphoacceptor site (FIG. 1). Arginine in the −1 position was selected over random amino acids and leucine at the −2 position was strongly selected, suggesting that a Leu-Arg-Thr motif would be a highly selected peptide substrate. Thr172 of AMPKα has a leucine at the −2 position which is conserved in AMPK orthologues from other species, including the yeast SNF1 protein (FIG. 1a), as well as a well-conserved arginine in the −1 position suggesting it would make an excellent in vitro substrate for LKB1. In addition, the previously mapped LKB1 phosphorylation sites in STRAD also conform to the LxT sequence.

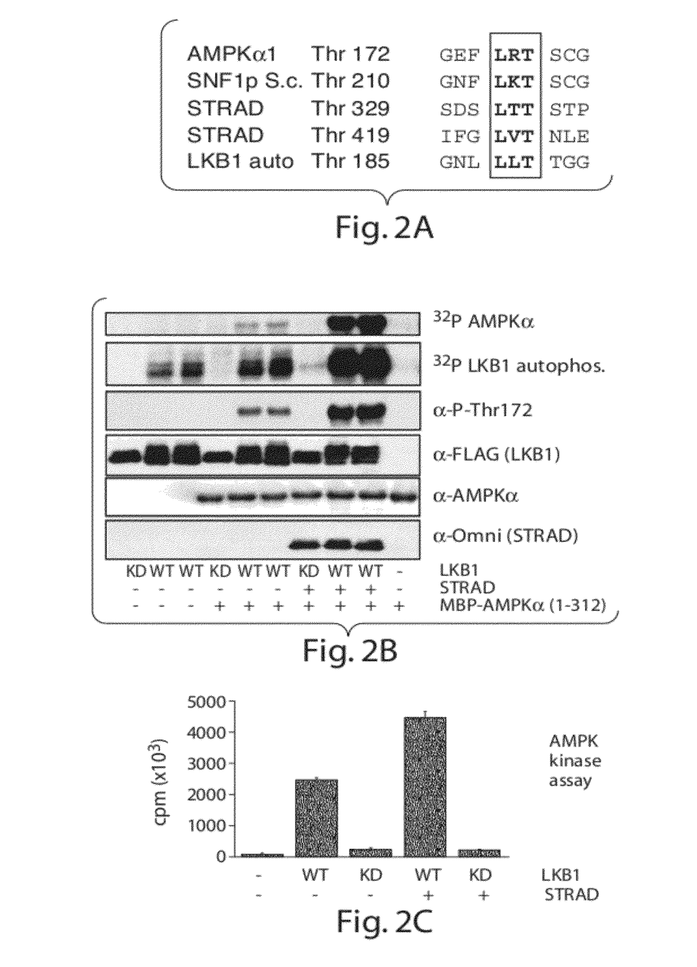

Given these observations, we investigated whether LKB1 would phosphorylate Thr172 of AMPK in vitro. As seen in FIG. 2b, wild-type but not kinase-dead LKB1 immunoprecipitated from mammalian cells efficiently phosphorylated a bacterially expressed maltose-binding protein (MBP) fusion product of the AMPKa catalytic subunit in vitro. Moreover, co-expression of LKB1 with STRAD led to a dramatic proportional increase in LKB1 autophosphorylation and trans-phosphorylation of MBP-AMPKa. Immunoblotting with phosphospecific AMPK Thr172 antisera confirmed that LKB1 phosphorylated this site in vitro. As seen in FIG. 2b, the level of immunoblotting with anti-phospho-Thr172 antibody was directly proportional to the amount of ³²P incorporation into recombinant APMK in a parallel radioactive in vitro kinase assay.

To examine whether in vitro phosphorylation of AMPK by LKB1 was sufficient to activate the bacterial MBP-AMPK fusion protein, we assayed the kinase activity of AMPKa using a specific peptide substrate (SAMS) (14). As previously reported (7), bacterial MBP-AMPK is inactive towards the peptide (FIG. 2C). In vitro phosphorylation of AMPK by wild-type LKB1 alone or STRAD-activated LKB1 induced AMPK kinase activity an average of 27 and 50 fold, respectively. Kinase-inactive LKB1 alone or coexpressed with STRAD was unable to activate AMPK.

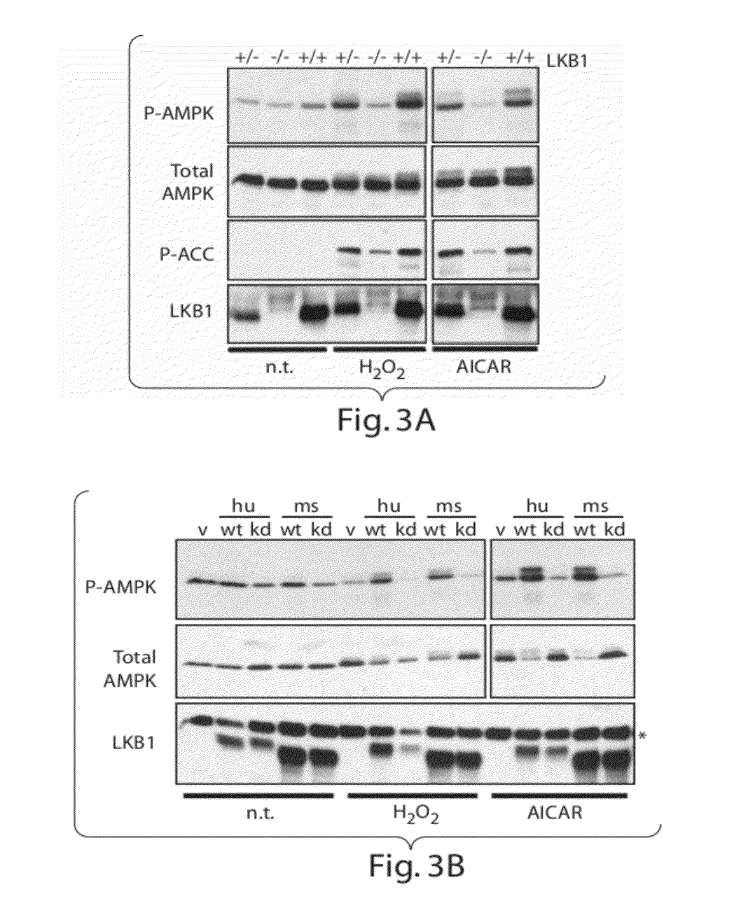

To rigorously test the requirement for LKB1 in AMPK activation in vivo, we derived LKB1-deficent murine embryonic fibroblasts (MEFs) from conditionally inactivated mice as previously described (15). Cells from littermate matched embryos were then stimulated as above and the response of AMPK examined. As seen in FIG. 3a, LKB1 null cells, but not wild-type or heterozygous controls, showed a complete loss of Thr172 phosphorylation in response to peroxide and AICAR stimulation. To determine if AMPK activity was accordingly downregulated in these cells, we examined the in vivo phosphorylation of one of its critical downstream substrates, acetyl CoA carboxylase (ACC). AMPK inactivates ACC through phosphorylation of serine 79, thereby stimulating fatty acid oxidations. Mirroring the level of phospho-AMPK, phosphorylation of ACC in response to both stimuli was nearly abolished in LKB1 null cells. It should be noted that there was still a small but reproducible amount of ACC phosphorylation in response to these stimuli in LKB1 null cells, suggesting the existence of other, minor compensating AMPKKs in MEFs, or the existence of other ACC kinases also activated by these stimuli. However, the dramatic reduction in phospho-AMPK and phospho-ACC indicates that LKB1 is the dominant AMPKK activity in these cells in response to the stimuli tested. To demonstrate that LKB1 loss itself, and not a secondary defect arising in these cells is responsible for impaired AMPK activation, we reintroduced wild-type and kinase dead LKB1 alleles into an immortalized LKB1 null MEF line by retrovirus. Indeed, wild-type but not kinase dead LKB1 expression potentiates AMPK activation and downstream phosphorylation of its targets (FIG. 3b, data not shown). Interestingly, despite the absence of LKB1 in these cells, kinase-dead LKB1 reduced the phosphorylation of AMPK and downstream ACC below the vector infected LKB1 null cells, suggesting that kinase dead LKB1 may block AMPK from being available as a substrate for other compensatory AMPKKs.

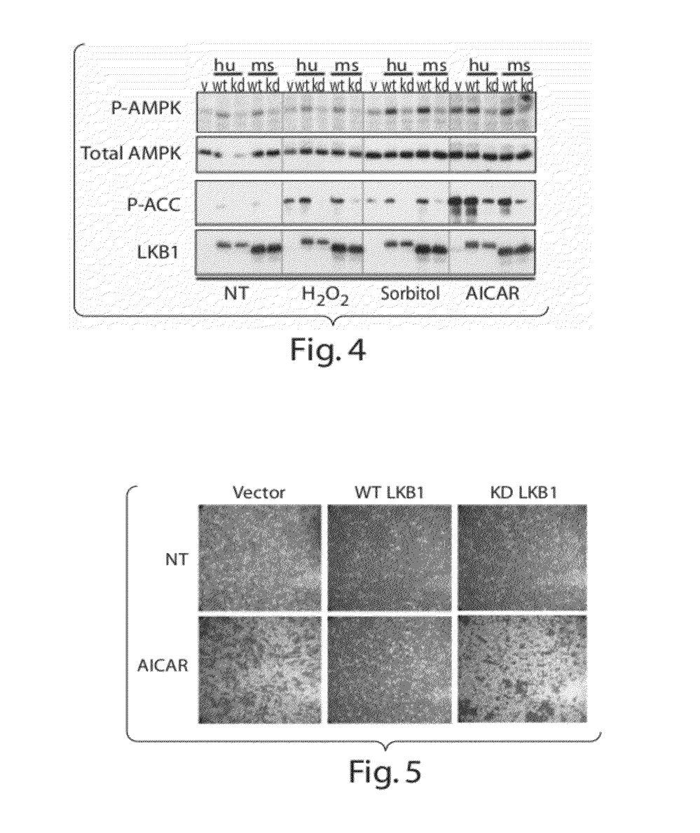

Given the requirement for LKB1 in AMPK activation in MEFs, we examined the ability of LKB1 to modulate AMPK activation in other cell types. As above, we used retroviruses to introduce wild-type or kinase-dead human and mouse LKB1 into a number of cell types. As seen in FIG. 4 in HT1080 human fibrosarcoma cells, kinase-dead LKB1 specifically inhibited AICAR, peroxide, and osmotic shock-induced Thr172 phosphorylation to levels below those seen in the vector-infected cells. Additionally, expression of wild-type LKB1 increased the basal and stimulated level of Thr172 phosphorylation (FIG. 3). As in the MEFs, phosphorylation of Ser79 of ACC is also increased basally and in response to all stimuli by wild-type LKB1 overexpression, indicating that AMPK activity is regulated in vivo. Similarly, expression of kinase dead LKB1 nearly abolishes the AMPK-induced phosphorylation of ACC in response to all three stimuli in HT1080 cells. Similar results were found in LLC-PK1 and IEC18 epithelial cells, as well as in HeLa cells, which are deficient in LKB1 protein due to promoter methylation (16) (data not shown).

To determine if LKB1 can mediate a biological response to a stimulus that activates the AMPKK/AMPK cascade we investigated whether LKB1 might modulate cell death under circumstances where AMPK would be activated. AMPK activation has been shown to lead to an inhibition of apoptosis in a number of cell types (17-20). Treatment of quiescent cells with AICAR protects them from glucocorticoid induced apoptosis and AICAR also protects astrocytes and endothelial cells from cell death in response to different stimuli. Furthermore, reduction of AMPK levels was recently shown to reduce cellular viability following glucose deprivation in a number of human tumor cell lines (20). We therefore examined the response of LKB1-deficient cells as compared to LKB1-reconstituted cells as above in their cellular response to apoptotic stimuli, including stimuli that are known to activate AMPK. Hela cells that were reconstituted with wildtype or kinase-dead LKB1 were treated with UV light or the AMP analogue AICAR. Strikingly, within 8 h of AICAR treatment, vector control and kinase-dead LKB1 expressing HeLa cells underwent extensive cell death, whereas their wild-type LKB1 expressing counterparts were nearly totally viable (FIG. 5a,b). In contrast, UV treatment killed all cell lines regardless of LKB1 status to a similar extent and with similar kinetics (FIG. 5c). Identical results were obtained with cells reconstituted with mouse or human LKB1. The extent of cell survival conferred by LKB1 reconstitution in the HeLa cells was quantified by MTT assay (FIG. 5b,c). These findings suggest that in cells expressing functional LKB1, AMPK signaling may provide a protective signal from some forms of cell death and in the absence of that protective signal, an apoptotic response is triggered. These results offer the provocative suggestion of a potential therapeutic window in which LKB1-deficient tumor cells might be acutely sensitive to AMP analogues or sensitized to cell death by other stimuli if treated in combination with AMPK activators.

Taken together, the results presented here suggest that LKB1 is a bona fide AMPKK and is the major AMPKK present in a number of cell types. These findings provide genetic and biochemical evidence that LKB1 is a critical regulator of AMPK in vivo. As potentially the central regulator of AMPK in vivo, LKB1 may play an unexpected role in multiple organ systems that mediate the diverse effects of AMPK on mammalian physiology. Importantly, AMPK has been shown to be a critical mediator of glucose uptake in skeletal muscle in mice (5) and AMPK kinase activity is stimulated by two major diabetes therapeutics (21,22). Therefore, identification of LKB1 as a major activator of AMPK in vivo may introduce a new set of potential avenues to exploit in the effort to boost AMPK activity in the treatment of diabetes. It will be critical to define the specific tissues in which LKB1 serves as the principal AMPKK and to determine which AMPK activating stimuli utilize LKB1 as opposed to other AMPKKs. While this manuscript was in preparation, it was reported that LKB1 can phosphorylate AMPK in vitro, based on the homology of LKB1 to three recently identified AMPKKs in S. cerevisiae (23). The presence of three functionally redundant AMPKKs in yeast (23,24) along with the small residual AMPK phosphorylation seen in LKB1-deficient cells suggests there will be additional mammalian AMPKKs.

Furthermore, through this work we have identified the first substrate of the LKB1 tumor suppressor that may mediate its downstream biological effects. Interestingly, we have found that LKB1-deficient cells are uniquely sensitized to death by the AMP analogue AICAR. These data suggest that LKB1/AMPK signaling plays a role in protection from apoptosis, and that stimuli that normally activate AMPK may lead to aberrant cell death in cells that are defective in AMPK signaling. LKB1/AMPK signaling may also play a role in other cellular responses to environmental stress. LKB1 deficient MEFs are resistant to passage-induced senescence (15). Recently, AMPK activity was found to increase in cells undergoing senescence, and artificial hyper-activation of AMPK promoted senescence in primary human fibroblasts suggesting that perhaps a loss of AMPK signaling promotes the immortalization of LKB1-deficient MEFs. Finally, defining the potential role of AMPK in tumorigenesis or as a potential regulator of cellular transformation or senescence will provide many further insights into the fundamental ties between energy metabolism, apoptosis, and aberrant cell growth.

### REFERENCES

1. Hardie, D. G., Scott, J. W., Pan, D. A., & Huson, E. R. Management of cellular energy by the AMP-activated protein kinase system. FEBS Letters 546, 1113-120 (2003).
2. Yoo, L. I., Chung, D. C., & Yuan, J. LKB1—a master tumor suppressor of the small intestine and beyond. Nat. Rev Cancer 2, 529-35 (2002).
3. Rutter, G. A., DaSilvaXavier, G.& Leclerc, I. Roles of 5′AMP-activated protein kinase in mammalian glucose homeostasis. Biochem J. 375 (Pt 1):1-16 (2003).
4. Minokoshi, Y., Kim, Y. B., Peroni, O. D., Fryer, L. G., Muller, C, Carling, D.& Kahn, B. B. Leptin stimulates fatty-acid oxidation by activating AMP-activated protein kinase. Nature 415, 339-43 (2002).
5. Mu, J., Brozinick, J. T. Jr, Valladares, O., Bucan, M. & Birnbaum, M. J. A role for AMP-activated protein kinase in contraction- and hypoxia-regulated glucose transport in skeletal muscle. Mol. Cell. 7, 1085-94 (2001).
6. Hawley, S. A, Davison, M., Woods, A., Davies, S. P., Beri, R. K., Carling, D., Hardie, D. G. Characterization of the AMP-activated protein kinase kinase from rat liver and identification of threonine 172 as the major site at which it phosphorylates AMPactivated protein kinase. J Biol. Chem. 271, 27879-87 (1996).
7. Hamilton, S. R., O'Donnell, J. B. Jr, Hammet, A., Stapleton, D., Habinowski, S. A., Means, A. R., Kemp, B. E.& Witters, L. A. AMP-activated protein kinase kinase: detection with recombinant AMPK alpha1 subunit. Biochem Biophys Res Commun. 293, 892-8 (2002)
8. Hawley, S. A., Seibert, M. A., Goldstein, E. G., Edelman, A. M., Carling, D., Hardie, D. G. 5′-AMP activates the AMP-activated protein kinase cascade, and Ca2+/calmodulin activates the calmodulin-dependent protein kinase I cascade, via three independent mechanisms. J. Biol. Chem. 270, 27186-91 (1995)
9. Martin, S. G.& St Johnston, D. A role for Drosophila LKB1 in anterior-posterior axis formation and epithelial polarity. Nature 421, 379-84 (2003)
10. Boudeau, J., Sapkota, G.& Alessi, D. R. LKB1, a protein kinase regulating cell proliferation and polarity. FEBS Lett. 546, 159-65 (2003)
11. Baas, A. F., Boudeau, J., Sapkota, G. P., Smit, L., Medema, R., Morrice, N. A., Alessi, D. R.& Clevers, H. C. Activation of the tumour suppressor kinase LKB1 by the STE20-like pseudokinase STRAD. EMBO J. 22, 3062-72 (2003).
12. Songyang, Z.& Cantley, L. C. The use of peptide library for the determination of kinase peptide substrates. Methods Mol. Biol. 87, 87-98 (1998).
13. Woods A, Vertommen D, Neumann D, Turk R, Bayliss J, Schlattner U, Wallimann T, Carling, D. & Rider, M. H. Identification of phosphorylation sites in AMP-activated protein kinase (AMPK) for upstream AMPK kinases and study of their roles by site-directed mutagenesis. J Biol. Chem. 278, 28434-42 (2003).
14. Davies, S. P., Carling, D., Hardie, D. G. Tissue distribution of the AMP-activated protein kinase, and lack of activation by cyclic-AMP-dependent protein kinase, studied using a specific and sensitive peptide assay. Eur J. Biochem. 186, 123-8 (1989).
15. Bardeesy, N., Sinha, M., Hezel, A. F., Signoretti, S., Hathaway, N. A., Sharpless, N. E., Loda, M., Carrasco, D. R. & DePinho, R. A. Loss of the Lkb1 tumour suppressor provokes intestinal polyposis but resistance to transformation. Nature 419, 162-7 (2002).
16. Tiainen, M., Ylikorkala, A.& Makela, T. P. Growth suppression by Lkb1 is mediated by a G(1) cell cycle arrest. Proc Natl Acad Sci USA. 96, 9248-51 (1999).
17. Stefanelli, C., Stanic, I., Bonavita, F., Flamigni, F., Pignatti, C., Guarnieri, C. & Caldarera, C. M. Inhibition of glucocorticoid-induced apoptosis with 5-aminoimidazole-4-carboxamide ribonucleoside, a cell-permeable activator of AMP-activated protein Biochem Biophys Res Commun. 243, 821-6 (1998).
18. Ido, Y., Carling, D.& Ruderman N. Hyperglycemia-induced apoptosis in human umbilical vein endothelial cells: inhibition by the AMP-activated protein kinase activation. Diabetes. 51, 159-67 (2002).
19. Culmsee, C., Monnig, J., Kemp, B. E. & Mattson M. P. AMP-activated protein kinase is highly expressed in neurons in the developing rat brain and promotes neuronal survival following glucose deprivation. J Mol Neurosci. 17, 45-58.
20. Kato, K., Ogura, T., Kishimoto, A., Minegishi, Y., Nakajima, N., Miyazaki, M., Esumi, H. Critical roles of AMP-activated protein kinase in constitutive tolerance of cancer cells to nutrient deprivation and tumor formation. Oncogene 21, 6082-90.
21. Zhou, G., Myers, R., Li, Y., Chen, Y., Shen, X., Fenyk-Melody, J., Wu, M., Ventre, J., Doebber, T., Fujii, N., Musi, N., Hirshman, M. F., Goodyear, L. J.& Moller, D. E. Role of AMP-activated protein kinase in mechanism of metformin action. J Clin Invest. 108, 1167-74 (2001).
22. Fryer, L. G., Parbu-Patel, A.& Carling, D. The Anti-diabetic drugs rosiglitazone and metformin stimulate AMP-activated protein kinase through distinct signaling pathways. J Biol. Chem. 277, 25226-32 (2002).
23. Hong S P, Leiper F C, Woods A, Carling D, Carlson M. Activation of yeast Snf1 and mammalian AMP-activated protein kinase by upstream kinases. Proc Natl Acad Sci USA. 100, 8839-43 (2003).
24. Sutherland, C. M., Hawley, S. A., McCartney, R. R., Leech, A., Stark, M. J., Schmidt, M. C., Hardie, D. G. Elm1p is one of three upstream kinases for the Saccharomyces cerevisiae SNF1 complex. Curr Biol. 13, 1299-305 (2003).
25. Wang, W., Yang, X., Lopez de Silanes, I., Carling, D., Gorospe, M. Increased AMP:ATP ratio and AMP-activated protein kinase activity during cellular senescence linked to reduced HuR function. J Biol. Chem. 278, 27016-23 (2003).
26. Shanahan, F., Seghezzi, W., Parry, D., Mahony, D. & Lees, E. Cyclin E associates with BAF155 and BRG1, components of the mammalian SWI-SNF complex, and alters the ability of BRG1 to induce growth arrest. Mol Cell Biol. 19, 1460-9 (1999).
27. Shaw, R. J., Paez, J. G., Curto, M., Yaktine, A., Pruitt, W. M., Saotome, I., O'Bryan, J. P., Gupta, V., Ratner, N., Der, C. J., Jacks, T.& McClatchey, A. I. The Nf2 tumor suppressor, merlin, functions in Rac-dependent signaling. Dev Cell. 1, 63-72 (2001).
28. Zhao, J. J., Gjoerup, O. V., Subramanian, R. R., Cheng, Y., Chen, W., Roberts, T. M. & Hahn, W. C. Human mammary epithelial cell transformation through the activation of phosphatidylinositol 3-kinase. Cancer Cell 3, 483-95 (2003).

### Example 2

### Introduction

We have examined the biochemical and biological relationship between LKB1 and mTOR regulation. Here, we report that LKB1 is required for repression of mTOR under low ATP conditions in cultured cells in an AMPK- and TSC2-dependent manner and that Lkb1-null MEFs and the hamartomatous gastrointestinal polyps from Lkb1-mutant mice show elevated signaling downstream of mTOR. These findings position aberrant mTOR activation at the nexus of these germline neoplastic conditions and suggest the use of mTOR inhibitors in the treatment of Peutz-Jeghers Syndrome.

### Methods

### Reagents and Cell Lines

Anti-phospho-AMPK (T172), anti-phospho-ACC(S79), anti-phospho-S6K1 (T389), anti-phospho-S6K1 (T421/S424), anti-phospho-GSK-3 (S9), anti-phospho ribosomal protein S6 (S235/236), anti-ribosomal protein S6, anti-eIF4E, anti-phospho-Akt (S473), anti-phospho-Erk (T202/Y204), anti-4E-BP1, cleaved PARP (mouse-specific) antibodies were obtained from Cell Signaling Technology (Beverly, Mass.). Anti-LKB1 antiserum was previously described (Bardeesy et al. (2002) Nature 419:162-167). Anti-Flag antibodies (M2 monoclonal and Flag polyclonal) were from Sigma. Anti-HA probe polyclonal antibody was from Santa Cruz Biotechnology (Santa Cruz, CA). AICAR was obtained from Toronto Research Chemicals (Downsview, ON, Canada). Glucose-free media, D-PBS (containing 1 g/L D-glucose) and dialyzed serum from Gibco/BRL. Rapamycin was from Calbiochem. 7-methyl GTP sepharose was obtained from Amersham Biosciences (Piscataway, N.J.). MBP-AMPKα (1-172) fusion protein was a generous gift of Dr. L. Witters and R. Hurley (Dartmouth Medical School, Hanover, N.H.). Active recombinant Akt and AMPK used in FIG. 2B were purchased from Upstate Biotechnology (Lake Placid, N.Y.). Constructs used: Flag-wild-type LKB1. Flag-kinase dead (K781) LKB1, Flag-tuberin, were described previously (Shaw et al. (2004), Proc. Natl. Acad. Sci. USA 101:3329-3335, Manning et al. (2002) Mol. Cell. July; 10(1):151-62). HA-4E-BP1 and HA-S6K1 were kind gifts of J. Blenis (Harvard Medical School, Boston, Mass.). pEBG-AMPK α1 T172A was a kind gift of Dr. L. Witters (Dartmouth Medical School, Hanover, N.H.). pcDNA3-AMPKα2 (K45R) kinase dead was a kind gift of M. Birnbaum (University of Pennsylvania Medical School, Philadelphia, Pa.). Littermate-derived LKB1+/+ and −/− MEFs were prepared as described previously (Bardeesy et al. (2002) Nature 419:162-167) TSC2−/− p53−/− and control p53−/− littermate MEFs were obtained from Dr. D. Kwiatkowski as previously described (Zhang et al (2003) J Clin Invest 112, 1223-1233). HeLa cells were obtained from ATCC (Manassas, Va.).

Cell culture

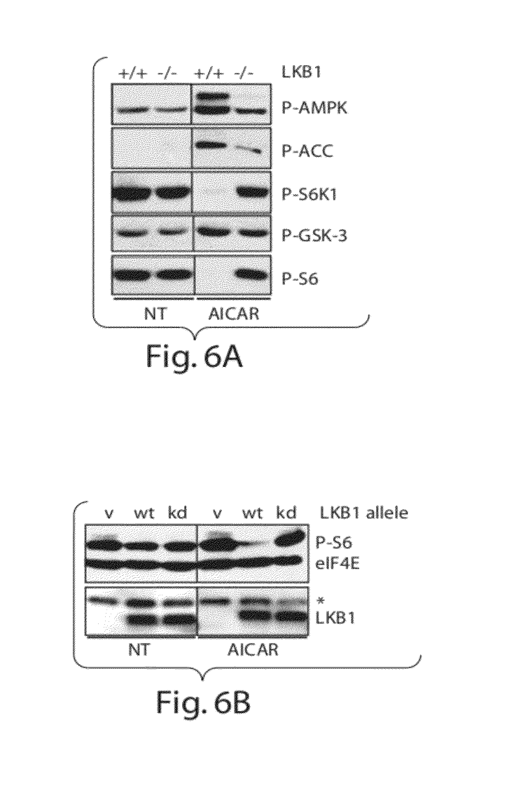

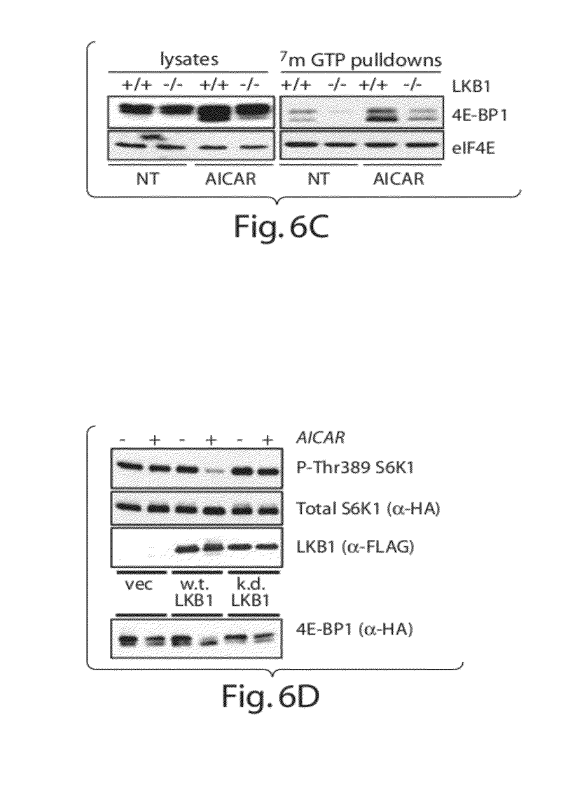

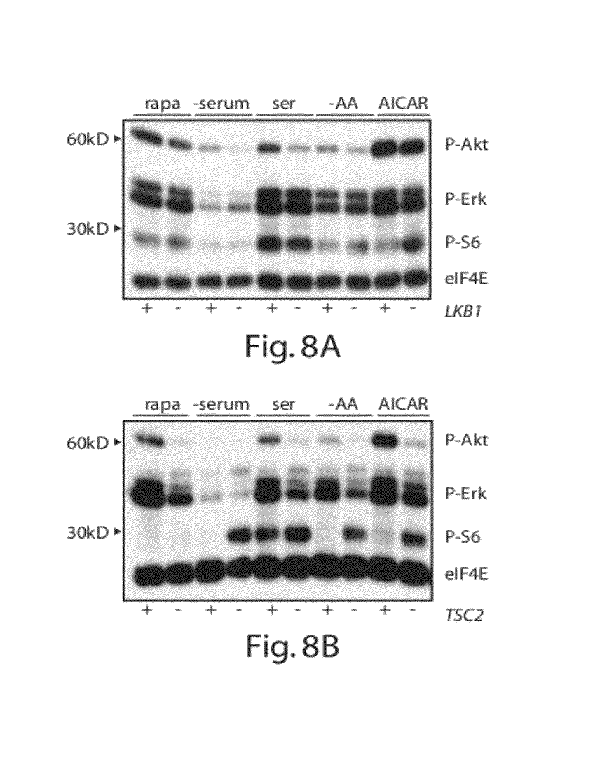

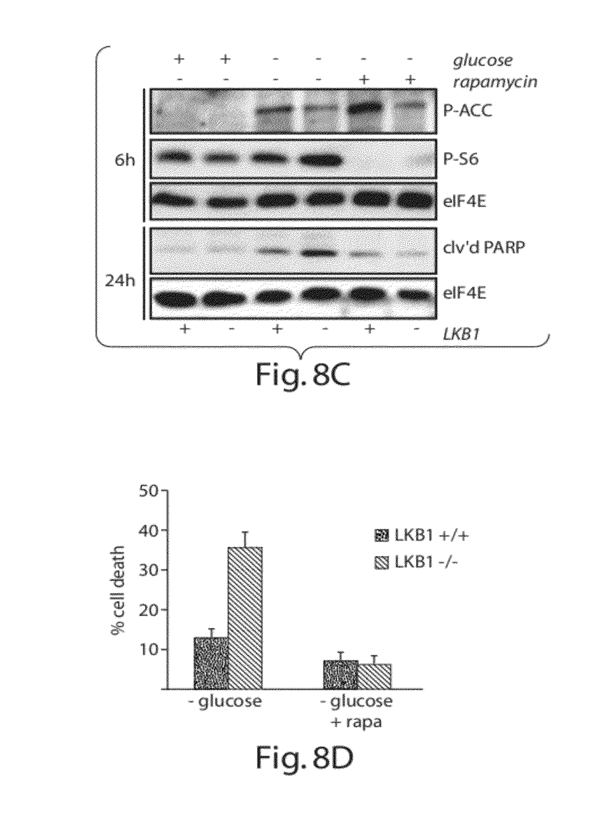

Cells were cultured and retrovirally infected as previously described (Shaw et al. (2004), Proc. Natl. Acad. Sci. USA 101:3329-3335). HeLa cells were transiently transfected using HeLaMonster transfection reagent according to manufacturer's suggestions (Minis, Madison, Wis.). Cells were serum-starved in DMEM without serum 14 h post-transfection, then 8 h later the media was replaced with fresh DMEM with 10% FBS or DMEM with 10% FBS and 2 mM AICAR. For the MEF experiments in FIG. 6: cells were plated at 1×10⁶ cells/10 cm dish, then the next day the media was replaced with fresh DMEM with 10% FBS or DMEM with 10% FBS and 2 mM AICAR. For the MEF experiments in FIG. 8A,B: 1×10⁶ cells of each genotype were plated and the next day serum-starved for 24 h. Cells were either lysed as such or their media replaced with fresh DMEM with 10% FBS, DMEM with 10% FBS and 2 mM AICAR, DMEM with 10% FBS and 20 nM rapamycin, or D-PBS with 10% dialyzed FBS for 90 mins. For FIG. 8C, D cells were plated and the next day placed in glucose-free DMEM with 10% dialyzed FBS, with or without 20 nM rapamycin. Cells were kept in glucose-free media with or without rapamycin for 24 h. For MTT assays cells were plated in 12 well dishes in triplicate for each condition. MTT assays were performed as previously described (Shaw et al. (2004), Proc. Natl. Acad. Sci. USA 101:3329-3335).

### Biochemistry

MEFs were lysed in boiling SDS-lysis buffer (10 mM Tris pH7.5, 100 mM NaCl, 1% SDS) after the indicated treatments. After trituration, lysates were equilibrated for protein levels using the BCA method (Pierce Biotechnology, Rockford, Ill.) and resolved on 6 to 12% SDS-PAGE gels, depending on the experiment. Gels were transferred to PVDF and western blotted according to the antibody manufacturer suggestions. Immunoprecipitations, kinase assays and 7-methyl GTP pulldowns were performed as previously described (Shaw et al. (2004), Proc. Natl. Acad. Sci. USA 101: 3329-3335, Manning et al. (2002) Mol. Cell. July; 10(1):151-62 (2002), Fingar et al. (2002) Oncogene 23: 3151-3171).

Mice Colony Monitoring and Tissue Isolation Lkb1+/− mice, maintained on an FVB/N genetic background, were monitored for the development of gastrointestinal polyps as described (Bardeesy et al. (2002) Nature 419:162-167). Mice with clinical signs of disease were euthanized and autopsied. The mean latency, distribution of polyps and polyp phenotype were comparable to previous studies (Bardeesy et al. (2002) Nature 419:162-167). Polyps and adjacent tissue were harvested immediately and either processed for histoligical analysis or snap frozen in liquid nitrogen for molecular studies. These samples were then placed frozen into Nunc tubes and homogenized in lysis buffer (20 mM Tris pH 7.5, 150 mM NaCl, 1 mM EDTA, 1 mM EGTA, 1% Triton X-100, 2.5 mM pyrophosphate, 50 mM NaF, 5 mM β-glycero-phosphate, 50 nM calyculin A, 1 mM Na₃VO4, 10 mM PMSF, 4 μg/ml leupeptin, 4 μg/ml pepstatin, 4 μg/ml aprotinin) on ice for 30s using a tissue homogenizer.

### Histology and Immunohistochemistry

Tissues were fixed in ice-cold 10% formalin overnight and embedded in paraffin. For immunohistochemistry, slides were deparaffinized in xylene and rehydrated sequentially in ethanol. Antigen retrieval using Antigen Unmasking Solution (Vector Labs, Burlingame, Calif.) was performed according to the manufacturer's instructions. Slides were quenched in hydrogen peroxide (0.3-3%) to block endogenous peroxidase activity and then washed in Automation Buffer (Biomeda, Foster City, Calif.). Slides were blocked in 5% normal serum for 1 hour at room temperature. Slides were incubated at 4° C. overnight with primary antibody diluted in blocking buffer. The avidin-biotin peroxidase complex method (Vector) was used and slides were counterstained with hematoxylin. Slides were dehydrated sequentially in ethanol, cleared with xylenes and mounted with Permount (Fisher Scientific, Hampton, N.H.). The anti-phospho ribosomal protein S6 antibody (S235/236) and anti-phospho-S6K1 (T421/5424) were diluted according to maufacturer's suggestions (Cell Signaling Technology, Beverly, Mass.).

### Results

To examine the role of AMPK in the regulation of mTOR signaling under physiological conditions, we evaluated mTOR activity in wild-type and Lkb1-deficient mouse embryonic fibroblasts (MEFs). In normal growth media, Lkb1+/+ and −/− MEFs show low AMPK activity as evidenced by lack of phosphorylation of the critical AMPK effector acetyl CoA carboxylase (ACC) (FIG. 6A). Under these conditions, phosphorylation of S6K1 and its substrate ribosomal S6 were readily detectable, consistent with high levels of mTOR activity. On the other hand, in response to treatment with 5-aminoimidizole-4-carboxamide riboside (AICAR), which serves as a cell permeable AMP mimetic (Hardie et al., (2003) FEBS Lett 546:113-120), AMPK was activated and S6K1 was inhibited in wild-type cells. Strikingly, in the Lkb1-deficient fibroblasts, AMPK activation by AICAR was severely attenuated, and levels of active S6K1 remained high (FIG. 6A). In contrast, growth factor signaling to GSK-3, reflective of Akt activity in these cells, was unaffected by AICAR-induced energy stress. We obtained similar results following treatment with the AMPK agonist phenformin. To ensure sustained elevation of mTOR activity was not a secondary consequence of LKB1 deficiency, we stably reconstituted immortalized Lkb1−/− MEFs with retroviruses encoding wild-type or kinase-dead Lkb1. As seen in FIG. 6B, wild-type but not kinase-dead Lkb1 restored the sensitivity of S6K1 activity to energy stress. A modest, but reproducible, effect on S6 phosphorylation was also seen under unstimulated conditions in wild-type Lkb1-reconstituted cells (FIG. 6B, lane 2).

To address whether other known effectors of mTOR were similarly deregulated by Lkb1 loss, or if S6K1 was specifically affected, we examined the regulation of 4E-BP1. The mTOR dependent-phosphorylation of 4E-BP1 is known to inhibit its association with eIF4E and the 5′-cap complex. The components of endogenous cap complexes can be assayed using 7-methyl GTP sepharose as an affinity reagent (Fingar et al. (2002) Oncogene 23:3151-3171). As seen in FIG. 6C (left panel), 4E-BP1 exists as a number of species with different mobilities on SDS-PAGE due to differences in phosphorylation, with the slowest mobility form being the hyper-phosphorylated form. In growing cells, 4E-BP1 is hyperphosphorylated and very little co-purifies in cap complexes, and that which does is restricted to the hypophosphorylated forms. Notably, treatment of wild-type MEFs with AICAR significantly increases the level of the hypophosphorylated 4E-BP1 and concomitantly leads to increased 4E-BP1 association with the cap complex. Interestingly, Lkb1-deficient cells displayed less 4E-BP1 associated with the cap complex, both under basal conditions and, more markedly, following AICAR treatment (FIG. 6C). This was also reflected in the near absence of hypophosphorylated 4E-BP1 following AICAR treatment in the total cell lysates (FIG. 6C top left panel).

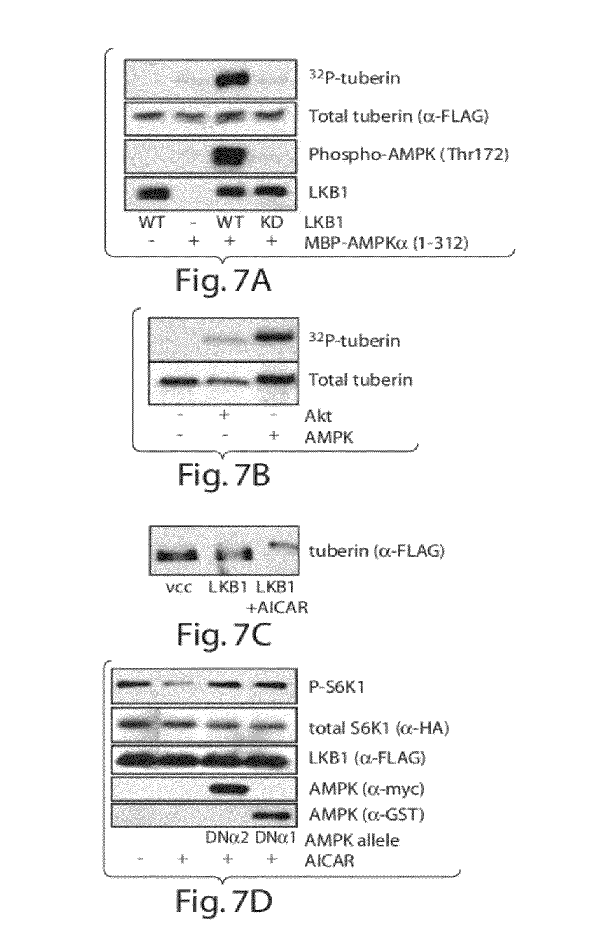

To determine whether LKB1-deficiency impacts broadly on mTOR signaling, we tested the LBK1-mTOR link in a distinct cell type of human origin—HeLa cells were selected as they are known to lack LKB1 expression (Tiainen et al. (1999) Proc Natl Acad Sci USA 96:9248-9251). Following transduction of wild-type or kinase-dead LKB1 along with HA-tagged S6K1 or 4E-BP1 constructs, we assayed mTOR activation of S6K1 by immunoblotting HA immunoprecipitates with anti-phosphoT389 antisera and mTOR phosphorylation of 4E-BP1 by mobility shift on SDS-PAGE. As seen in FIG. 6D, introduction of wild-type, but not kinase-dead, LKB1 into HeLa cells restored AICAR-dependent inhibition of S6K1 and 4E-BP1 phosphorylation in response to energy stress indicating that LKB1-dependent mTOR regulation is conserved in humans and in different cell types. Recent studies have demonstrated that the TSC2 tumor suppressor protein, tuberin, is a direct target of AMPK and a critical mediator of AMPK-dependent inhibition of mTOR signaling (Inoki et al. (2003) Cell 115:577-590). We examined whether LKB1 can regulate tuberin phosphorylation via AMPK, and whether AMPK itself is critical for the effects of LKB1 in downregulating mTOR signaling. First, we examined the ability of purified recombinant AMPK pre-activated in vitro with LKB1 to directly phosphorylate tuberin. As seen in FIG. 7A, AMPK activated by wild-type LKB1 directly phosphorylated tuberin in vitro. Moreover, we noticed that when tuberin was stoichiometrically phosphorylated by AMPK, it underwent a mobility shift when resolved on low percentage SDS-PAGE gels (FIG. 7B). This mobility shift in tuberin was not observed when tuberin was phosphorylated by active recombinant Akt, even at levels of tuberin phosphorylation comparable to those induced by AMPK. To assess this phosphorylation in vivo, we co-transfected HeLa cells with FLAG-tagged tuberin with or without LKB1. We then examined the mobility of tuberin on SDS-PAGE following treatment with AICAR. HeLa cells expressing wild-type LKB1 induced a significant reduction in tuberin mobility after treatment of the cells with AICAR (FIG. 7C), suggesting LKB1 induced AMPK activation and subsequent phosphorylation of tuberin. AICAR treatment in the absence of LKB1 expression had no effect on tuberin mobility in HeLa cells.

In order to more rigorously examine the requirement of AMPK for LKB1-mediated inhibition of mTOR signaling, we introduced wild-type LKB1 into HeLa cells with or without two different dominant-negative AMPK constructs—a kinase-dead alpha2 allele and a non-activatible T172A alpha1 allele (Crute et al. (1998) J Biol Chem 273:35347-35354). As seen in FIG. 7D, co-expression of either dominant-negative AMPK alpha allele blocked the ability of LKB1 to downregulate S6K1 in AICAR-treated HeLa cells.

We next wished to determine whether other known stresses that inhibit mTOR activity might also function through LKB1/AMPK. For these experiments, we compared the response of littermate matched Lkb1+/+ and −/− MEFs to littermate matched p53-deficient Tsc2+/+ and −/− MEFs. Tsc2−/− MEFs have been reported to display aberrantly high levels of mTOR signaling in response to a wide-variety of cellular stresses (Zhang et al. (2003) J Clin Invest 112:1223-1233). As seen in FIG. 8A, Lkb1−/− cells failed to down-regulate S6K1 activity following AICAR treatment, as shown above. However, LKB1-deficient cells quite effectively down-regulated S6K1 activity following amino acid and growth factor deprivation. In contrast, in Tsc2−/− cells, S6K1 was not inhibited in response to any of these stimuli (FIG. 8A, B). Importantly, rapamycin inhibited S6K1 activity in all cell types. In order to examine whether other mitogenic signaling pathways are affected by the loss of Lkb1, we used phospho-specific antibodies that recognize activated forms of Erk and Akt. We found that neither Erk nor Akt activation was enhanced in Lkb1-deficient cells following any of the aforementioned cellular stresses. In fact, Akt activation was reduced in LKB1-deficient cells as compared to wild-type controls under several conditions, including serum deprivation, serum stimulation, and AICAR treatment (FIG. 8). Similarly, as seen previously (Kwiatkowski et al. (2002) Proc Natl Acad Sci USA 100: 12923-12928; Jaeschke et al. (2002) J Cell Biol 159:217-224), Akt activity was dramatically attenuated in the Tsc2-deficient cells following all stimuli, a phenomenon which has previously been attributed to a negative feedback loop inhibiting PI3K/Akt signaling in a number of systems (Garami et el. (2003) Mol Cell 11:1457-1466).

Given these similarities in intracellular signaling, we examined whether Lkb1−/−MEFs might share any biological properties with TSC2−/− MEFs. One property of LKB1-deficient cells that we previously characterized was their sensitivity, relative to wild-type cells, to undergo apoptosis following treatment with AICAR or other AMPK agonists (Shaw et al. (2004) Proc Natl Acad. Sci. USA 101:3329-3335). Notably, Tsc2−/− MEFs were recently found to undergo apoptosis under conditions of glucose deprivation, which also serves to activate AMPK (Inoki et al., Inoki et al. (2003) Cell 115, 577-590). Interestingly, this property of Tsc2-deficient MEFs was rescued by concurrent treatment with rapamycin. We therefore investigated whether glucose deprivation would also selectively lead to the apoptosis of Lkb1-deficient MEFs and whether this effect could be rescued by rapamycin treatment. First we characterized that AMPK signaling and mTOR was aberrant in LKB1−/− cells following glucose deprivation as seen in FIG. 8C. As expected, in the LKB1−/− cells, phospho-S6 was elevated and phospho-ACC was decreased, indicating a defect in AMPK signaling in these cells following glucose deprivation. Using caspase activation-as detected by PARP cleavage in lysates-as a measure of apoptosis (Shaw et al. (2004) Proc Nall Acad Sci USA 101: 3329-3335; Inoki et al. (2003) Cell 115, 577-590), we observed that Lkb1−/− MEFs showed elevated sensitivity to apoptosis induced by glucose withdrawal as compared to their wild-type counterparts, and that rapamycin potently inhibited this apoptotic phenotype (FIG. 8C). The PARP cleavage in lysates was mirrored in direct assays of cell viability (FIG. 8D). These results confirm the sensitivity of LKB1−/− MEFs to glucose-deprivation induced cell death and the suppression of this effect by rapamycin.

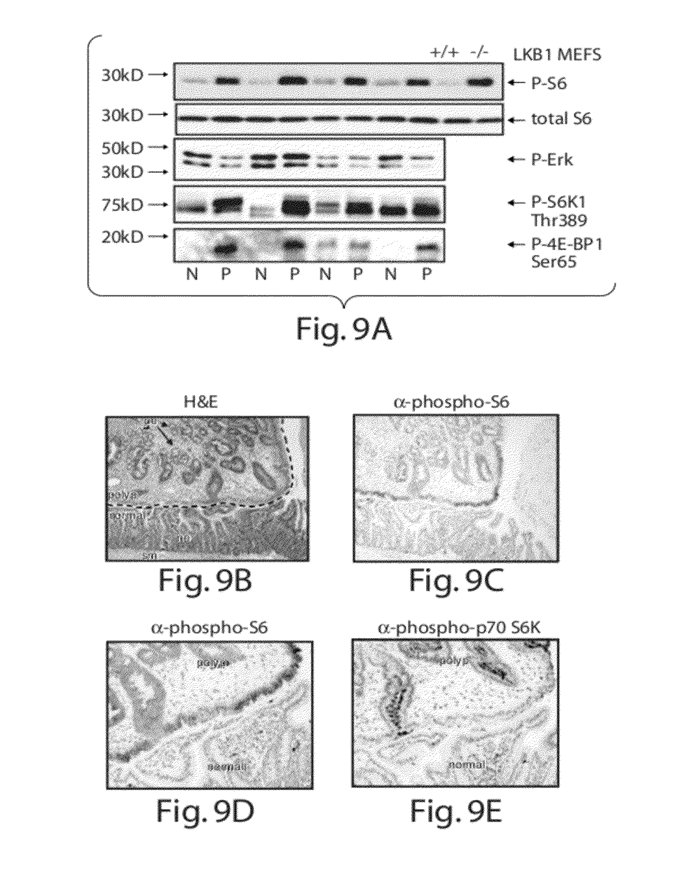

The regulation of mTOR by LKB1 in vivo is intriguing in light of the established role of mTOR activation in the pathogenesis of human tumors. We examined the potential role of mTOR deregulation in the pathogenesis of the hamartomatous polyps that characterize LKB1-deficiency in both humans and mice. We investigated the status of the mTOR pathway in hamartomatous gastrointestinal polyps arising in Lkb1+1-mice. Western blot analyses of lysates from polyps and adjacent gastrointestinal epithelium revealed a prominent elevation in phospho-S6 and phospho-S6K1 (Thr389) levels in all polyps analyzed (FIG. 9A). Furthermore, we detected elevated levels of phospho-4E-BP1 (Ser65), indicating that mTOR-dependent signaling was elevated in the polyps. This property appears to be specific to Lkb1-deficiency rather than secondary to the deregulated growth state of the polyps since other growth-associated markers, such as phospho-Erk (FIG. 9A) and phospho-Akt levels did not show consistent differences between polyp and adjacent normal tissue. These results were extended by immunohistochemical analyses. While both the normal gastrointestinal epithelium adjacent to the polyp tissue and the polyp stroma showed minimal immuno-reactivity to anti-phospho-S6 antisera, the polyp epithelium showed intense cytoplasmic staining (FIG. 9B-D). The activation of S6K1 in the polyps was further confirmed and extended by additional immunohistochemical studies that revealed strong staining for phospho-S6K1 (Thr421/Ser424) specifically in the polyp epithelium (FIG. 9E). The staining pattern showed both nuclear and cytoplasmic localization consistent with increased phosphorylation of both the p70 and p85 S6K isoforms. Importantly, the epithelial cells within the polyp that contain elevated levels of phospho-S6 and -S6K1 are thought to be the neoplastic component of the polyp (Hemminki et al. (1997) Nat Genet. 15:87-90; Wang et al. (1998) J Pathol 188:9-13; Entiusk et al. (2001) J Clin Pathol 54:126-131; Bardeesy et al. (2002) Nature 419:162-167.

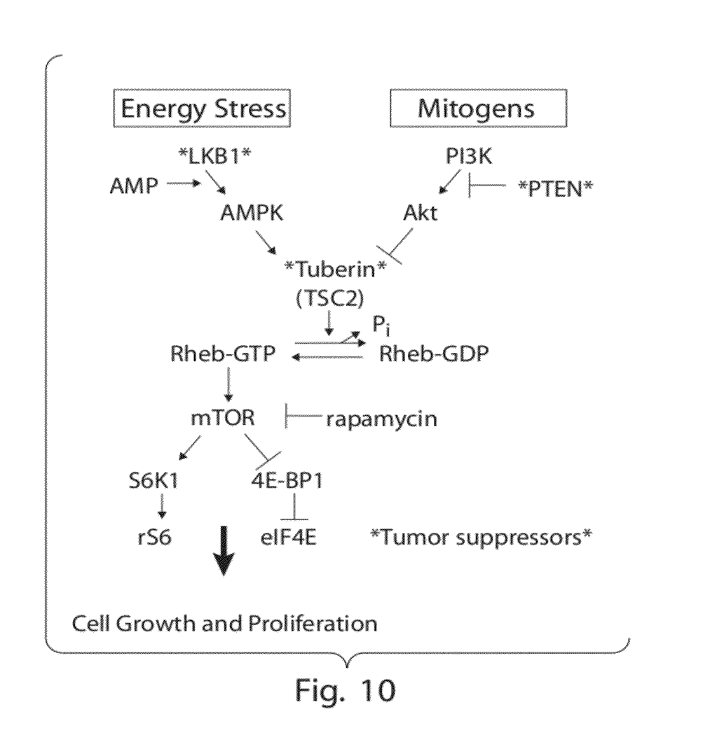

The development of hamartomas is a stereotypical feature of a number of clinically related human tumor disorders, including Peutz-Jeghers syndrome, Cowden's disease (and related Pten-deficiency disorders), and tuberous sclerosis complex (Gomez, et al., 1999; Cantley and Neel, 1999; Devroede et al., 1988). As previously demonstrated for Pten- and Tsc2-deficient tumors in vivo, LKB1-deficient tumors are shown here to exhibit elevated levels of S6K1 activity and phospho-4EBP1, indicative of hyperactivation of mTOR signaling, establishing hyperactivate mTOR signaling as a common theme across these distinct tumor suppressor disorders (FIG. 10). In this study, we further demonstrate that, in cell culture-based systems, LKB1 modulation of mTOR signaling is mediated through LKB1-dependent phosphorylation of AMPK in response to energy stress and that phosphorylation of tuberin by AMPK is a likely mechanism to explain the effects on mTOR signaling. These results provide strong biochemical and genetic evidence that endogenous AMPK mediates mTOR inhibition in response to decreased intracellular ATP.

### Discussion

The development of hamartomas is a stereotypical feature of a number of clinically related human tumor disorders, including Peutz-Jeghers syndrome, Cowden's disease (and related Pten-deficiency disorders), and tuberous sclerosis complex (Gomez, 1999; Cantley and Neel, 1999; Devroede et al., 1988). As previously demonstrated for Pten- and Tsc2-deficient tumors in vivo, LKB1-deficient tumors are shown here to exhibit elevated levels of S6K1 activity and phospho-4EBP1, indicative of hyperactivation of mTOR signaling, establishing hyperactivate mTOR signaling as a common theme across these distinct tumor suppressor disorders (FIG. 5). In this study, we further demonstrate that, in cell culture-based systems, LKB1 modulation of mTOR signaling is mediated through LKB1-dependent phosphorylation of AMPK in response to energy stress and that phosphorylation of tuberin by AMPK is a likely mechanism to explain the effects on mTOR signaling. These results provide strong biochemical and genetic evidence that endogenous AMPK mediates mTOR inhibition in response to decreased intracellular ATP.

Consistent with mTOR as a key effector in the biology of LKB1, Tsc2−/− and Lkb1−/− MEFs share a number of properties that are uncharacteristic of cells lacking most tumor suppressors. Both cell types are hypersensitive to apoptosis induced by energy stress, and this adverse response can be rescued by treatment with the mTOR inhibitor rapamycin. Both also display attenuated Akt activation under most conditions, which is in stark contrast to loss of Pten, which causes hyper-activation of Akt. Aberrant mTOR signaling might also explain two major molecular markers of LKB1 loss previously characterized. VEGF has been shown to be upregulated in embryos and MEFs deficient for LKB1 (Ylikorkala et al., 2001; Bardeesy et al., 2002), or TSC2 (El-Hashemite et al., 2003; Brugarolas et al., 2003). Of note, the increase in VEGF protein levels in LKB1-deficient MEFs is not associated with increased VEGF mRNA levels (Bardeesy et al., 2002), a finding consistent with data demonstrating VEGF to be one of several genes whose oncogene-induced recruitment of mRNA to polyribosomes is critically governed by mTOR signaling (Rajasekhar et al., 2003). Another marker of LKB1 loss is the expression of IGFBP5, the single most upregulated transcript in LKB1+/− mouse GI polyps as compared to normal surrounding tissue. IGFBP5 mRNA levels are also aberrantly high in the LKB1-deficient MEFs (Bardeesy et al., 2002). The transcription of IGFBP5 has previously been shown to be critically dependent on mTOR signaling and is inhibited by rapamycin (Duan et al., 1999). Furthermore, IGFBP5 translation is also dependent on mTOR (Rajasekhar et al., 2003). Thus, hyper-activation of mTOR signaling in the LKB1-deficienct MEFs, polyps, and embryos is likely to explain their previously noted elevated levels of VEGF and IGFBP5.

The multiple overlapping clinical features between Peutz-Jeghers syndrome and Cowden's disease (CD), caused us to originally hypothesize that LKB1, like PTEN, might negatively regulate PI3-kinase signaling (Cantley and Neel, 1999). However, we have observed in a number of biological settings that Akt activity is not increased in LKB1-deficient cells, and if anything is decreased (see FIG. 3A). This reduced Akt activation in the LKB1-deficient cells can also account for the previous observation of decreased phospho-GSK3 (Ser9), in LKB1-deficient MEFs (Ossipova et al., 2003). These results suggest that the common biochemical basis underlying the PJS and CD syndromes is not due to a shared activation of PI3K/Akt signaling but rather to upregulation of mTOR signaling in both LKB1- and Pten-deficient cells.

One significant question that remains is whether AMPK is the only kinase downstream of LKB1 that can down-regulate mTOR via TSC2 phosphorylation or other mechanisms. LKB1 has been demonstrated to activate 11 AMPK-related kinases in an energy stress-independent manner (Lizcano et al., 2004; Sakamoto et al., 2004). In different cellular contexts, these kinases may phosphorylate TSC2 or other shared substrates with AMPK. Indeed we have observed that LKB1 can regulate basal mTOR signaling under non-stressed conditions (e.g., FIG. 6C). Additional work will be needed to determine whether any of the other AMPK-related kinases can regulate TSC2/mTOR, and to determine which kinase contributes to mTOR regulation in the pathogenesis of Peutz-Jeghers polyposis. Additionally, phosphorylation of TSC2 may not be the only way in which AMPK, or related kinases, negatively regulate mTOR. The recent report that AMPK can directly phosphorylate mTOR itself, and does so under conditions of energy stress, offers another possible inhibitory signal (Cheng et al., 2004). Therefore, while the lack of response to energy stress in TSC2-deficient cells argues that tuberin is important; it may not be the only target of AMPK in mTOR regulation.

Recently, LKB1 has been demonstrated to be necessary and sufficient for polarity of single intestinal epithelial cells (Baas, 2004). LKB1 kinase activity was shown to be necessary for this effect, although which of its 13 downstream effector kinases might play a role in this process has not been delineated. Moreover, LKB1 is the mammalian homolog of Par-4 in C. elegans which was one of the original par mutants identified in a screen for early embryonic polarity and partitioning defects (Watts et al., 2000). The Drosophila ortholog of LKB1 is also essential for early embryonic polarity (Martin and St. Johnston, 2003) and some of its role in that process is likely due to its activation of the Par-1 kinase homolog, which is one of the 11 kinases related to AMPK that have recently been demonstrated to be activated by LKB1 (Lizcano et al., 2004). Thus LKB1 uniquely regulates both central metabolism, through AMPK, and at least some aspects of cell polarity via Par-1 homologs. Therefore, disruption of LKB1 in the gastrointestinal epithelium would potentially simultaneously result in aberrant mTOR activation and defects in cell polarity. Whether LKB1-mediated inhibition of mTOR plays a role in the establishment of cell polarity remains to be determined.

However, it has been demonstrated that in yeast, TOR signaling is critically involved in cell polarity (Loewith et al., 2002; reviewed in Harris and Lawrence, 2003).

Together, with the results shown here, it is now evident that dysregulated activation of mTOR is a common biochemical feature of several autosomal dominant syndromes characterized by the occurrence of hamartomas. These results also suggest that rapamycin and its analogs may be useful for the treatment of polyps arising in PJS patients and possibly in the subset of lung adenocarcinomas that lack LKB1. The fact that rapamycin can promote survival of LKB1-deficient cells under conditions of energy stress, as previously reported for TSC-deficient cells, suggests that there may be certain cell types that are sensitive to growth inhibition by rapamycin, while others may be refractory. The biological response to mTOR inhibition via rapamycin (growth arrest vs. apoptosis) is also known to depend on other factors such as p53 status (Huang et al., 2003). While rapamycin might allow LKB1-deficient cells to survive in response to energy stress in vitro, it is unlikely that these cells would continue to proliferate in the presence of rapamycin. Of course, additional studies will be required to validate these possibilities, but the similarity of the polyps developed in the LKB1 heterozygous mice to those in PJS patients suggests that these mice represent an ideal tool to examine this potential therapeutic regiment.

LKB1 mutations are associated with the Peutz-Jeghers syndrome (PJS), consisting of pigmentation anomalies, benign gastrointestinal polyps (hamartomas) and predisposition to a range of malignant tumor types. This study forges a biochemical and genetic link from Lkb1 to mTOR signaling via the sequential activation of AMPK and the TSC2 tumor suppressor. This inter-relationship, particularly hyperactive mTOR signaling, provides a rational explanation for the shared features of three human disorders characterized by the development of hamartomas (PJS, Cowden's Disease, and Tuberous Sclerosis Complex). mTOR inhibitors including rapamycin analogs which are currently under clinical trials for a number of cancers, may be effective in the treatment and possible prevention of Peutz-Jeghers polyps and sporadic tumors that show LKB1 loss.

### REFERENCES

Aguirre, A. J., Bardeesy, N., Sinha, M., Lopez, L., Tuveson, D. A., Horner, J., Redston, M. S., and DePinho, R. A. (2003). Activated Kras and Ink4a/Arf deficiency cooperate to produce metastatic pancreatic ductal adenocarcinoma. Genes Dev 17, 3112-3126.
Baas, A. F., Kuipers, J., van der Wel, N. N., Batlle, E., Koerten, H. K., Peters, P. J., and Clevers, H. C. (2004). Complete polarization of single intestinal epithelial cells upon activation of LKB1 by STRAD. Cell 116, 457-466.
Bardeesy, N., Sinha, M., Hezel, A. F., Signoretti, S., Hathaway, N. A., Sharpless, N. E., Loda, M., Carrasco, D. R., and DePinho, R. A. (2002). Loss of the Lkb1 tumour suppressor provokes intestinal polyposis but resistance to transformation. Nature 419, 162-167.
Boudeau, J., Sapkota, G., and Alessi, D. R. (2003). LKB1, a protein kinase regulating cell proliferation and polarity. FEBS Lett 546, 159-165.
Brugarolas, J. B., Vazquez, F., Reddy, A., Sellers, W. R., and Kaelin, W. G., Jr. (2003). TSC2 regulates VEGF through mTOR-dependent and -independent pathways. Cancer Cell 4, 147-158.
Cantley, L. C., and Neel, B. G. (1999). New insights into tumor suppression: PTEN suppresses tumor formation by restraining the phosphoinositide 3-kinase/AKT pathway. Proc Natl Acad Sci USA 96, 4240-4245.
Carretero, J., Medina, P. P., Pio, R., Montuenga, L. M., and Sanchez-Cespedes, M. (2004). Novel and natural knockout lung cancer cell lines for the LKB1/STK11 tumor suppressor gene. Oncogene 23, 4037-4040.
Cheng, S. W., Fryer, L. G., Carling, D., and Shepherd, P. R. (2004). Thr2446 is a novel mammalian target of rapamycin (mTOR) phosphorylation site regulated by nutrient status. J Biol Chem 279, 15719-15722.
Crute, B. E., Seefeld, K., Gamble, J., Kemp, B. E., and Witters, L. A. (1998). Functional domains of the alpha1 catalytic subunit of the AMP-activated protein kinase. J Biol Chem 273, 35347-35354.
Dennis, P. B., Jaeshke, A., Saitoh, M., Fowler, B., Kozma, S. C., and Thomas, G. (2001). Mammalian TOR: a homeostatic ATP sensor. Science 294, 1102-1105.
Devroede, G., Lemieux, B., Masse, S., Lamarche, J., and Herman, P. S. (1988). Colonic hamartomas in tuberous sclerosis. Gastroenterology 94, 182-188.
Duan, C., Liimatta, M. B., and Bottum, O. L. (1999). Insulin-like growth factor (IGF)-I regulates IGF-binding protein-5 gene expression through the phosphatidylinositol 3-kinase, protein kinase B/Akt, and p70 S6 kinase signaling pathway. J Biol Chem 274, 37147-37153.
El-Hashemite, N., Walker, V., Zhang, H., and Kwiatkowski, D. J. (2003). Loss of Tsc1 or Tsc2 induces vascular endothelial growth factor production through mammalian target of rapamycin. Cancer Res 63, 5173-5177.
Entius, M. M., Keller, J. J., Westerman, A. M., van Rees, B. P., van Velthuysen, M. L., de Goeij, A. F., Wilson, J. H., Giardiello, F. M., and Offerhaus, G. J. (2001). Molecular genetic alterations in hamartomatous polyps and carcinomas of patients with Peutz-Jeghers syndrome. J Clin Pathol 54, 126-131.
Fingar, D. C., Salama, S., Tsou, C., Harlow, E., and Blenis, J. (2002). Mammalian cell size is controlled by mTOR and its downstream targets S6K1 and 4EBP1/eIF4E. Genes Dev 16, 1472-1487.
Fingar, D. C., and Blenis, J. (2004). Target of rapamycin (TOR): an integrator of nutrient and growth factor signals and coordinator of cell growth and cell cycle progression. Oncogene 23, 3151-3171.
Garami, A., Zwartkruis, F. J. T., Nobukuni, T., Joaquin, M., Roccio, M., Stocker, H., Kozma, S. C., Hafen, E., Bos, J. L., and Thomas, G. (2003). Insulin activation of Rheb, a mediator of mTOR/S6K/4E-BP signaling, is inhibited by TSC1 and 2. Mol Cell 11, 1457-1466.
Gomez, M. R., Sampson, J. R., and Whittemore, V. H. (1999). Tuberous Sclerosis, 3rd edn (New York, N.Y., Oxford University Press).
Hardie, D. G., Scott, J. W., Pan, D. A., and Hudson, E. R. (2003). Management of cellular energy by the AMP-activated protein kinase system. FEBS Lett 546, 113-120.
Harris, T. E., and Lawrence, J. C., Jr. (2003). TOR signaling. Sci STKE 2003, re15.
Hawley, S. A., Boudeau, J., Reid, J. L., Mustard, K. J., Udd, L., Makela, T. P., Alessi, D. R., and Hardie, D. G. (2003). Complexes between the LKB1 tumor suppressor, STRADalpha/beta and MO25 alpha/beta are upstream kinases in the AMP-activated protein kinase cascade. J Biol 2, 28.
Hemminki, A., Tomlinson, I., Markie, D., Jarvinen, H., Sistonen, P., Bjorkqvist, A. M., Knuutila, S., Salovaara, R., Bodmer, W., Shibata, D., et al. (1997). Localization of a susceptibility locus for Peutz-Jeghers syndrome to 19p using comparative genomic hybridization and targeted linkage analysis. Nat Genet. 15, 87-90.
Huang, S., Shu, L., Dilling, M. B., Easton, J., Harwood, F. C., Ichijo, H., and Houghton, P. J. (2003). Sustained activation of the JNK cascade and rapamycin-induced apoptosis are suppressed by p53/p21 (Cip1). Mol Cell 11, 1491-1501.
Inoki, K., Li, Y., Zhu, T., Wu, J., and Guan, K. L. (2002). TSC2 is phosphorylated and inhibited by Akt and suppresses mTOR signalling. Nat Cell Biol 4, 648-657.
Inoki, K., Zhu, T., and Guan, K. L. (2003). TSC2 mediates cellular energy response to control cell growth and survival. Cell 115, 577-590.
Jishage, K., Nezu, J., Kawase, Y., Iwata, T., Watanabe, M., Miyoshi, A., Ose, A., Habu, K., Kake, T., Kamada, N., et al. (2002). Role of Lkb1, the causative gene of Peutz-Jegher's syndrome, in embryogenesis and polyposis. Proc Natl Acad Sci USA 99, 8903-8908.
Jaeschke, A., Hartkamp, J., Saitoh, M., Roworth, W., Nobukuni, T., Hodges, A., Sampson, J., Thomas, G., and Lamb, R. (2002). Tuberous sclerosis complex tumor suppressor-mediated S6 kinase inhibition by phosphatidylinositide-3-OH kinase is mTOR independent. J Cell Biol 159, 217-224.

Jorgensen, S. B., Viollet, B., Andreelli, F., Frosig, C., Birk, J. B., Schjerling, P., Vaulont, S., Richter, E. A., and Wojtaszewski, J. F. (2004). Knockout of the alpha2 but not alpha1 5′-AMP-activated protein kinase isoform abolishes 5-aminoimidazole-4-carboxamide-1-beta-4-ribofuranosidebut not contraction-induced glucose uptake in skeletal muscle. J Biol Chem 279, 1070-1079.
Kemp, B. E., Stapleton, D., Campbell, D. J., Chen, Z. P., Murthy, S., Walter, M., Gupta, A., Adams, J. J., Katsis, F., van Denderen, B., et al. (2003). AMP-activated protein kinase, super metabolic regulator. Biochem Soc Trans 31, 162-168.
Kenerson, H. L., Aicher, L. D., True, L. D., and Yeung, R. S. (2002). Activated mammalian target of rapamycin pathway in the pathogenesis of tuberous sclerosis complex renal tumors. Cancer Res 62, 5645-5650.

Kimura, N., Tokunaga, C., Dalal, S., Richardson, C., Yoshino, K., Hara, K., Kemp, B. E., Witters, L. A., Mimura, O., and Yonezawa, K. (2003). A possible linkage between AMP-activated protein kinase (AMPK) and mammalian target of rapamycin (mTOR) signalling pathway. Genes Cells 8, 65-79.
Kwiatkowski, D. J., Zhang, H., Bandura, J. L., Heiberger, K. M., Glogauer, M., el-Hashemite, N., and Onda, H. (2002). A mouse model of TSC1 reveals sex-dependent lethality from liver hemangiomas, and up-regulation of p70S6 kinase activity in TSC1 null cells. Hum Mol Genet. 11, 525-534.
Kwon, C. H., Zhu, X., Zhang, J., and Baker, S. J. (2003). mTor is required for hypertrophy of Pten-deficient neuronal soma in vivo. Proc Natl Acad Sci USA 100, 12923-12928.
Lizcano, J. M., Goransson, O., Toth, R., Deak, M., Morrice, N. A., Boudeau, J., Hawley, S. A., Udd, L., Makela, T. P., Hardie, D. G., and Alessi, D. R. (2004). LKB1 is a master kinase that activates 13 kinases of the AMPK subfamily, including MARK/PAR-1. Embo J 23, 833-843.
Loewith, R., Jacinto, E., Wullschleger, S., Lorberg, A., Crespo, J. L., Bonenfant, D., Oppliger, W., Jenoe, P., and Hall, M. N. (2002). Two TOR complexes, only one of which is rapamycin sensitive, have distinct roles in cell growth control. Mol Cell 10, 457-468.

Manning, B. D., and Cantley, L. C. (2003). United at last: the tuberous sclerosis complex gene products connect the phosphoinositide 3-kinase/Akt pathway to mammalian target of rapamycin (mTOR) signalling. Biochem Soc Trans 31, 573-578.
Martin, S. G., and St Johnston, D. (2003). A role for Drosophila LKB1 in anterior-posterior axis formation and epithelial polarity. Nature 421, 379-384.
McManus, E. J., and Alessi, D. R. (2002). TSC1-TSC2: a complex tale of PKB-mediated S6K regulation. Nat Cell Biol 4, E214-216.
Miyoshi, H., Nakau, M., Ishikawa, T. 0., Seldin, M. F., Oshima, M., and Taketo, M. M. (2002). Gastrointestinal hamartomatous polyposis in Lkb1 heterozygous knockout mice. Cancer Res 62, 2261-2266.
Neshat, M. S., Mellinghoff, I. K., Tran, C., Stiles, B., Thomas, G., Petersen, R., Frost, P., Gibbons, J. J., Wu, H., and Sawyers, C. L. (2001). Enhanced sensitivity of PTEN-deficient tumors to inhibition of FRAP/mTOR. Proc Natl Acad Sci USA 98, 10314-10319.
Onda, H., Crino, P. B., Zhang, H., Murphey, R. D., Rastelli, L., Gould-Rothberg, B. E., and Kwiatkowski, D. J. (2002). TSC2 null murine neuroepithelial cells are a model for human tuber giant cells, and show activation of an mTOR pathway. Mol Cell Neurosci 21, 561-574.
Ossipova, O., Bardeesy, N., DePinho, R. A., and Green, J. B. (2003). LKB1 (XEEK1) regulates Wnt signalling in vertebrate development. Nat Cell Biol 5, 889-894.
Podsypanina, K., Lee, R. T., Politis, C., Hennessy, I., Crane, A., Puc, J., Neshat, M., Wang, H., Yang, L., Gibbons, J., et al. (2001). An inhibitor of mTOR reduces neoplasia and normalizes p70/56 kinase activity in PTEN+/− mice. Proc Natl Acad Sci USA 98, 10320-10325.
Rajasekhar, V. K., Viale, A., Socci, N. D., Wiedmann, M., Hu, X., and Holland, E. C. (2003). Oncogenic Ras and Akt signaling contribute to glioblastoma formation by differential recruitment of existing mRNAs to polysomes. Mol Cell 12, 889-901.
Ramaswamy, S., Nakamura, N., Vazquez, F., Batt, D. B., Perera, S., Roberts, T. M., and Sellers, W. R. (1999). Regulation of G1 progression by the PTEN tumor suppressor protein is linked to inhibition of the phosphatidylinositol 3-kinase/Akt pathway. Proc Natl Acad Sci USA 96, 2110-2115.
Rossi, D. J., Ylikorkala, A., Korsisaari, N., Salovaara, R., Luukko, K., Launonen, V., Henkemeyer, M., Ristimaki, A., Aaltonen, L. A., and Makela, T. P. (2002). Induction of cyclooxygenase-2 in a mouse model of Peutz-Jeghers polyposis. Proc Natl Acad Sci USA 99, 12327-12332.
Sakamoto, K., Goransson, O., Hardie, D. G., and Alessi, D. R. (2004). Activity of LKB1 and AMPK-related kinases in skeletal muscle; effects of contraction, phenformin and AICAR. Am J Physiol Endocrinol Metab.
Sanchez-Cespedes, M., Parrella, P., Esteller, M., Nomoto, S., Trink, B., Engles, J. M., Westra, W. H., Herman, J. G., and Sidransky, D. (2002). Inactivation of LKB1/STK11 is a common event in adenocarcinomas of the lung. Cancer Res 62, 3659-3662.
Scott, J. W., Hawley, S. A., Green, K. A., Anis, M., Stewart, G., Scullion, G. A., Norman, D. G., and Hardie, D. G. (2004). CBS domains form energy-sensing modules whose binding of adenosine ligands is disrupted by disease mutations. J Clin Invest 113, 274-284.
Shamji, A. F., Nghiem, P., and Schreiber, S. L. (2003). Integration of growth factor and nutrient signaling: implications for cancer biology. Mol Cell 12, 271-280.
Shaw, R. J., Kosmatka, M., Bardeesy, N., Hurley, R. L., Witters, L. A., DePinho, R. A., and Cantley, L. C. (2004). The tumor suppressor LKB1 kinase directly activates AMP-activated kinase and regulates apoptosis in response to energy stress. Proc Natl Acad Sci USA 101, 3329-3335.
Shi, Y., Gera, J., Hu, L., Hsu, J., Bookstein, R., Li, W., and Lichtenstein, A. (2002). Enhanced sensitivity of multiple myeloma cells containing PTEN mutations to CCI-779. Cancer Res 62, 5027-5034.
Stambolic, V., Suzuki, A., delaPompa, J. L., Brothers, G. M., Mirtsos, C., Sasaki, T., Ruland, J., Penninger, J. M., Sioderovski, D. P., and Mak, T. W. (1998). Negative regulation of PKB/Akt-dependent cell survival by the tumor suppressor PTEN. Cell 95, 29-39.
Stein, S. C., Woods, A., Jones, N. A., Davison, M. D., and Carling, D. (2000). The regulation of AMP-activated protein kinase by phosphorylation. Biochem J 345 Pt 3, 437-443.
Suzuki, A., Kusakai, G., Kishimoto, A., Lu, J., Ogura, T., and Esumi, H. (2003). ARKS suppresses the cell death induced by nutrient starvation and death receptors via inhibition of caspase 8 activation, but not by chemotherapeutic agents or UV irradiation. Oncogene 22, 6177-6182.
Sun, H., Lesche, R., Li, D., Liliental, J., Zhang, H., Gao, J., Gavrilova, N., Mueller, B., Liu, X., and Wu, H. (1999). PTEN modulates cell cycle progression and cell survival by regulating phosphatidylinositol 3,4,5-triphosphate and Akt/protein kinase B signaling pathway. Proc Natl Acad Sci USA 96, 6199-6204.
Suzuki, A., Kusakai, G., Kishimoto, A., Minegichi, Y., Ogura, T., and Esumi, H. (2003). Induction of cell-cell detachment during glucose starvation through F-actin conversion by SNARK, the fourth member of the AMP-activated protein kinase catalytic subunit family. Biochem Biophys Res Commun 311, 156-161.
Tiainen, M., Ylikorkala, A., and Makela, T. P. (1999). Growth suppression by Lkb1 is mediated by a G(1) cell cycle arrest. Proc Natl Acad Sci USA 96, 9248-9251.
Tee, A. R., Fingar, D. C., Manning, B. D., Kwiatkowski, D. J., Cantley, L. C., and Blenis, J. (2002). Tuberous sclerosis complex-1 and -2 gene products function together to inhibit mammalian target of rapamycin (mTOR)-mediated downstream signaling. Proc Natl Acad Sci USA 99, 13571-13576.
Viollet, B., Andreelli, F., Jorgensen, S. B., Perrin, C., Geloen, A., Flamez, D., Mu, J., Lenzner, C., Baud, O, Bennoun, M., et al. (2003). The AMP-activated protein kinase alpha2 catalytic subunit controls whole-body insulin sensitivity. J Clin Invest 111, 91-98.
Wang, Z. J., Ellis, I., Zauber, P., Iwama, T., Marchese, C., Talbot, I., Xue, W. H., Yan, Z. Y., and Tomlinson, I. (1999). Allelic imbalance at the LKB1 (STK11) locus in tumours from patients with Peutz-Jeghers' syndrome provides evidence for a hamartoma-(adenoma)-carcinoma sequence. J Pathol 188, 9-13.
Watts, J. L., Morton, D. G., Bestman, J., and Kemphues, K. J. (2000). The C. elegans par-4 gene encodes a putative serine-threonine kinase required for establishing embryonic asymmetry. Development 127, 1467-1475.
Woods, A., Johnstone, S. R., Dickerson, K., Leiper, F. C., Fryer, L. G., Neumann, D., Schlattner, U., Wallimann, T., Carlson, M., and Carling, D. (2003). LKB1 is the upstream kinase in the AMP-activated protein kinase cascade. Curr Biol 13, 2004-2008.
Ylikorkala, A., Rossi, D. J., Korsisaari, N., Luukko, K., Alitalo, K., Henkemeyer, M., and Makela, T. P. (2001). Vascular abnormalities and deregulation of VEGF in Lkb1-deficient mice. Science 293, 1323-1326.
Zhang, H., Cicchetti, G., Onda, H., Koon, H. B., Asrican, K., Bajraszewski, N., Vazquez, F., Carpenter, C. L., and Kwiatkowski, D. J. (2003). Loss of Tsc1/Tsc2 activates mTOR and disrupts PI3K-Akt signaling through downregulation of PDGFR. J Clin Invest 112, 1223-1233.

Other aspects of the invention will be clear to the skilled artisan and need not be repeated here. Each reference cited herein is incorporated by reference in its entirety.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

We claim:

1-70. (canceled)

71. A method for treating cancer, comprising
administering to a subject having a cancer having reduced or absent LKB1 activity an effective amount of phenformin, wherein the reduction of LKB1 activity is due to a mutation or deletion of the LKB1 gene.

72. The method of claim 1, further comprising subjecting the cancer of the subject or cells thereof to a cell death stimulus.

73. A method for promoting apoptosis of cells having reduced or absent LKB1 activity, comprising
contacting the cells with phenformin, wherein the reduction of LKB1 activity is due to a mutation or deletion of the LKB1 gene.

74. A method of treating cancer comprising
administering to a subject having a cancer having reduced or absent LKB1 activity an effective amount of phenformin, wherein the reduction of LKB1 activity is due to a mutation or deletion of the LKB1 gene.
